SEQUENCE LISTING (60 sequences)

SEQ ID NO: 1 - protein, 326 aa, human
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGMVFSCSVMHEALHNHYTQKSLSLSLGK

SEQ ID NO: 2 - DNA, 978 bp, human
gcctccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtggagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 3 - protein, 326 aa, human
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 4 - DNA, 2009 bp, human
agctttctggggcgagccgggcctgactttggctttggggcagggagtgggctaaggtgaggcaggtggcgccagccaggtgcacacccaatgcccgtgagcccagacactggaccctgcctggaccctcgtggatagacaagaaccgaggggcctctgcgcctgggcccagctctgtcccacaccgcggtcacatggcaccacctctcttgcagcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccaggcaggcacaggctgggtgcccctaccccaggcccttcacacacaggggcaggtgcttggctcagacctgccaaaagccatatccgggaggaccctgcccctgacctaagccgaccccaaaggccaaactgtccactccctcagctcggacaccttctctcctcccagatccgagtaactcccaatcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaaggtgggacccgcggggtatgagggccacatggacagaggccggctcggcccaccctctgccctgggagtgaccgctgtgccaacctctgtccctacagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgccacggccggcaagcccccgctccccaggctctcggggtcgcgtgaggatgcttggcacgtaccccgtgtacatacttcccaggcacccagcatggaaataaagcacccagcgctgccctgggcccctgcgagactgtgatggttctttccgtgggtcaggccgagtctgaggcctgagtggcatgagggaggcagagtgggtc

SEQ ID NO: 5 - protein, 326 aa, human
STKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - DNA, 2028 bp, human
agctttctggggcaggccgggcctgactttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctcccggcaccacaggctggatgcccctaccccaggccctgcgcatacagggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgagggaggcagagcgggtcccactgtccccacactgg

SEQ ID NO: 7 - DNA, 6058 bp, artificial sequence
  vector
acgcgttgacattgattattgactagttattaatagtaatcaattacggggtcattagttcatagcccatatatggagttccgcgttacataacttacggtaaatggccccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagaattctgttgggctcgcggttgattacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgaccggcttggtaccgagctcggatccggaccatcatgaagtggagctgggttattctcttcctcctgtcagtaactgccggcgtccactcccaggttcaggtccagcagtctggggctgagctggcaagaccttgggcttcagtgaagttgtcctgcaaggcttctggctacaattttaatagttactggatgcagtgggtaaaacagaggcctggacagggtctggaatggattggggctatttatcctggagatggtgatactagctacactcagaagttcaggggcaaggccacattgactgcagataaatcctccagcacagcctacatgcaactcagcagcttggcatctgaggactctgcggtctattactgtgcaagacgtacggtaggaggctactttgactactggggccaaggcaccactctcacagtctcctcagcctccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctcccggcaccacaggctggatgcccctaccccaggccctgcgcatacagggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccatccatcacactggcggccgctcgagcatgcatctagaacttgtttattgcagcttataatggttacaaataaagcaatagcatcacaaatttcacaaataaagcatttttttcactgcattctagttgtggtttgtccaaactcatcaatgtatcttatcatgtctggatcgatcccgccatggtatcaacgccatatttctatttacagtagggacctcttcgttgtgtaggtaccgctgtattcctagggaaatagtagaggcaccttgaactgtctgcatcagccatatagcccccgctgttcgacttacaaacacaggcacagtactgacaaacccatacacctcctctgaaatacccatagttgctagggctgtctccgaactcattacaccctccaaagtcagagctgtaatttcgccatcaagggcagcgagggcttctccagataaaatagcttctgccgagagtcccgtaagggtagacacttcagctaatccctcgatgaggtctactagaatagtcagtgcggctcccattttgaaaattcacttacttgatcagcttcagaagatggcggagggcctccaacacagtaattttcctcccgactcttaaaatagaaaatgtcaagtcagttaagcaggaagtggactaactgacgcagctggccgtgcgacatcctcttttaattagttgctaggcaacgccctccagagggcgtgtggttttgcaagaggaagcaaaagcctctccacccaggcctagaatgtttccacccaatcattactatgacaacagctgttttttttagtattaagcagaggccggggacccctgggcccgcttactctggagaaaaagaagagaggcattgtagaggcttccagaggcaacttgtcaaaacaggactgcttctatttctgtcacactgtctggccctgtcacaaggtccagcacctccataccccctttaataagcagtttgggaacgggtgcgggtcttactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaaggagctcccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 8 - protein, 137 aa, human
MKWSWVILFLLSVTAGVHSQVQVQQSGAELARPWASVKLSCKASGYNFNSYWMQWVKQRPGQGLEWIGAIYPGDGDTSYTQKFRGKATLTADKSSSTAYMQLSSLASEDSAVYYCARRTVGGYFDYWGQGTTLTVSS

SEQ ID NO: 9 - protein, 12 aa, human
ESKYGPPCPSCP

SEQ ID NO: 10 - protein, 110 aa, human
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 11 - protein, 107 aa, human
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - DNA, 6057 bp, artificial sequence
  vector
acgcgttgacattgattattgactagttattaatagtaatcaattacggggtcattagttcatagcccatatatggagttccgcgttacataacttacggtaaatggccccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagaattctgttgggctcgcggttgattacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgaccggcttggtaccgagctcggatccggaccatcatgaagtggagctgggttattctcttcctcctgtcagtaactgccggcgtccactcccaggttcaggtccagcagtctggggctgagctggcaagaccttgggcttcagtgaagttgtcctgcaaggcttctggctacaattttaatagttactggatgcagtgggtaaaacagaggcctggacagggtctggaatggattggggctatttatcctggagatggtgatactagctacactcagaagttcaggggcaaggccacattgactgcagataaatcctccagcacagcctacatgcaactcagcagcttggcatctgaggactctgcggtctattactgtgcaagacgtacggtaggaggctactttgactactggggccaaggcaccactctcacagtctcctcagcctccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccaggcaggcacaggctgggtgcccctaccccaggcccttcacacacaggggcaggtgcttggctcagacctgccaaaagccatatccgggaggaccctgcccctgacctaagccgaccccaaaggccaaactgtccactccctcagctcggacaccttctctcctcccagatccgagtaactcccaatcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccatccatcacactggcggccgctcgagcatgcatctagaacttgtttattgcagcttataatggttacaaataaagcaatagcatcacaaatttcacaaataaagcatttttttcactgcattctagttgtggtttgtccaaactcatcaatgtatcttatcatgtctggatcgatcccgccatggtatcaacgccatatttctatttacagtagggacctcttcgttgtgtaggtaccgctgtattcctagggaaatagtagaggcaccttgaactgtctgcatcagccatatagcccccgctgttcgacttacaaacacaggcacagtactgacaaacccatacacctcctctgaaatacccatagttgctagggctgtctccgaactcattacaccctccaaagtcagagctgtaatttcgccatcaagggcagcgagggcttctccagataaaatagcttctgccgagagtcccgtaagggtagacacttcagctaatccctcgatgaggtctactagaatagtcagtgcggctcccattttgaaaattcacttacttgatcagcttcagaagatggcggagggcctccaacacagtaattttcctcccgactcttaaaatagaaaatgtcaagtcagttaagcaggaagtggactaactgacgcagctggccgtgcgacatcctcttttaattagttgctaggcaacgccctccagagggcgtgtggttttgcaagaggaagcaaaagcctctccacccaggcctagaatgtttccacccaatcattactatgacaacagctgttttttttagtattaagcagaggccggggacccctgggcccgcttactctggagaaaaagaagagaggcattgtagaggcttccagaggcaacttgtcaaaacaggactgcttctatttctgtcacactgtctggccctgtcacaaggtccagcacctccataccccctttaataagcagtttgggaacgggtgcgggtcttactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaaggagctcccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 13 - protein, 98 aa, human
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 14 - protein, 12 aa, human
ERKCCVECPPCP

SEQ ID NO: 15 - protein, 109 aa, human
APPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 16 - protein, 107 aa, human; identical to SEQ ID NO: 11

SEQ ID NO: 17 - protein, 467 aa, murine
MERHWIFLLLLSVTAGVHSQVQLQQSGAELARPGASVKMSCKASYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 18 - DNA, 1569 bp, murine
gaattcccctctccacagacactgaaaactctgactcaacatggaaaggcctggatctttctactcctgttgtcagtaactgcaggtgtccactcccaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgagctcagcacccacaaaactctcaggtccaaagagacacccacactcatctccatgcttcccttgtataaataaagcacccagcaatgcctgggaccatgtaaaaaaaaaaaaaaaaggaattc

SEQ ID NO: 19 - protein, 235 aa, murine
MDFQVQIFSFLLISASVIISRGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 20 - DNA, 943 bp, murine
gaattcccaaagacaaaatggattttcaagtgcagattttcagcttcctgctaatcagtgcctcagtcataatatccagaggacaaattgttctcacccagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaaccgggctgatactgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagagacaaaggtcctgagacgccaccaccagctcccagctccatcctatcttcccttctaaggtcttggaggcttccccacaagcgcttaccactgttgcggtgctctaaacctcctcccacctccttctcctcctcctccctttccttggcttttatcatgctaatatttgcagaaaatattcaataaagtgagtctttgccttgaaaaaaaaaaaaaa

SEQ ID NO: 21 - DNA, 819 bp, murine
aagcttatgaatatgcaaatcctctgaatctacatggtaaatataggtttgtctataccacaaacagaaaaacatgagatcacagttctctctacagttactgagcacacaggacctcaccatgggatggagctgtatcatcctcttcttggtagcaacagctacaggtaaggggctcacagtagcaggcttgaggtctggacatatatatgggtgacaatgacatccactttgcctttctctccacaggtgtccactcccaggtccagctgcaacagtctggggctgaactcgcaagacctggggcctcagtgaagatgtcctgcaaggcttctggctacacgtttactaggtacacgatgcactgggtaaaacagaggcctggacaaggtttggaatggattggatacattaaccctagccgtggatatactaattacaatcagaagttcaaggacaaggccacactgactacagacaaatcttccagcacagcctacatgcaactgagcagcctgacatctgaggactccgcagtctattactgtgcaagatattatgatgatcattactgtctcgactactggggccaaggcaccactttgacagtctcctcaggtgagtccttacaacctctctcttctattcagcttaaatagattttactgcatttgttgggggggaaatgtgtgtatctgaatttcaggtcatgaaggactagggacaccttgggagtcagaaagggtcattgggagcccgggctgatgcagacagacatcctcagctcccagacttcatggccagagatttataggatcc

SEQ ID NO: 22 - protein, 15 aa, murine
MGWSCIILFLVATAT

SEQ ID NO: 23 - protein, 123 aa, murine
GVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 24 - DNA, 617 bp, murine
aagcttatgaatatgcaaatcctctgaatctacatggtaaatataggtttgtctataccacaaacagaaaaacatgagatcacagttctctctacagttactgagcacacaggacctcaccatgggatggagctgtatcatcctcttcttggtagcaacagctacaggtaaggggctcacagtagcaggcttgaggtctggacatatatatgggtgacaatgacatccactttgcctttctctccacaggtgtccactcccaaattgttctcacccagtctccagcaatcatgtctgcatctccaggggaaaaggtcaccatgacatgcagtgccagctcaagtgtaagttacatgaactggtaccagcagaagtcaggcacctcccccaaaagatggatttatgacacatcaaaactggcttctggagtaccggctcacttcaggggcagtgggtctgggacctcttactctctcacaatctcagggatggaagctgaagatgccgcaacttattactgccagcagtggtcaagtaacccattcacgttcggatctggtacaaagttggaaatcaaacgtgagtagaatttaaactttgcttcctcagttggatcc

SEQ ID NO: 25 - protein, 15 aa, murine
MGWSCIILFLVATAT

SEQ ID NO: 26 - protein, 110 aa, murine
GVHSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIK

SEQ ID NO: 27 - DNA, 2026 bp, human
ggatcctctagattgagctttctggggcaggccaggcctgaccttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcctccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccaggcaggcacaggctgggtgcccctaccccaggcccttcacacacaggggcaggtgcttggctcagacctgccaaaagccatatccgggaggaccctgcccctgacctaagccgaccccaaaggccaaactgtccactccctcagctcggacaccttctctcctcccagatccgagtaactcccaatcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgaggaattcagatctggatcc

SEQ ID NO: 28 - DNA, 2757 bp, artificial sequence
  murine/human construct
aagcttatgaatatgcaaatcctctgaatctacatggtaaatataggtttgtctataccacaaacagaaaaacatgagatcacagttctctctacagttactgagccgtacggccgccaccatggattgggtctggaccttgctattcctcttgtcagtaactgcaggtgtccactcacaggtccagctgcaacagtctggggctgaactcgcaagacctggggcctcagtgaagatgtcctgcaaggcttctggctacacgtttactaggtacacgatgcactgggtaaaacagaggcctggacaaggtttggaatggattggatacattaaccctagccgtggatatactaattacaatcagaagttcaaggacaaggccacactgactacagacaaatcttccagcacagcctacatgcaactgagcagcctgacatctgaggactccgcagtctattactgtgcaagatattatgatgatcattactgtctcgactactggggccaaggcaccactttgacagtctcctcaggtgagtccttacaacctctctcttctattcagcttaaatagattttactgcatttgttgggggggaaatgtgtgtatctgaatttcaggtcatgaaggactagggacaccttgggagtcagaaagggtcattgggagcccgggctgatgcagacagacatcctcagctcccagacttcatggccagagatttataggatcctctagattgagctttctggggcaggccaggcctgaccttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcctccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccaggcaggcacaggctgggtgcccctaccccaggcccttcacacacaggggcaggtgcttggctcagacctgccaaaagccatatccgggaggaccctgcccctgacctaagccgaccccaaaggccaaactgtccactccctcagctcggacaccttctctcctcccagatccgagtaactcccaatcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgaggaattcagatctggatcc

SEQ ID NO: 29 - protein, 138 aa, murine
MDWVWTLLFLLSVTAGVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 30 - protein, 98 aa, human; identical to SEQ ID NO: 13

SEQ ID NO: 31 - protein, 12 aa, human
ERKCCVECPPCP

SEQ ID NO: 32 - protein, 109 aa, human; identical to SEQ ID NO: 15

SEQ ID NO: 33 - protein, 107 aa, human; identical to SEQ ID NO: 11

SEQ ID NO: 34 - DNA, 10554 bp, artificial sequence
  vector
gtgaccaatacaaaacaaaagcgcccctcgtaccagcgaagaaggggcagagatgccgtagtcaggtttagttcgtccggcggcgggggatctgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagaattctgttgggctcgcggttgattacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgaccggcttggtaccgagctcggatcctctagattgagctttctggggcaggccaggcctgaccttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcctccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccaggcaggcacaggctgggtgcccctaccccaggcccttcacacacaggggcaggtgcttggctcagacctgccaaaagccatatccgggaggaccctgcccctgacctaagccgaccccaaaggccaaactgtccactccctcagctcggacaccttctctcctcccagatccgagtaactcccaatcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgaggaattcagatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccccaggaagctcctctgtgtcctcataaaccctaacctcctctacttgagaggacattccaatcataggctgcccatccaccctctgtgtcctcctgttaattaggtcacttaacaaaaaggaaattgggtaggggtttttcacagaccgctttctaagggtaattttaaaatatctgggaagtcccttccactgctgtgttccagaagtgttggtaaacagcccacaaatgtcaacagcagaaacatacaagctgtcagctttgcacaagggcccaacaccctgctcatcaagaagcactgtggttgctgtgttagtaatgtgcaaaacaggaggcacattttccccacctgtgtaggttccaaaatatctagtgttttcatttttacttggatcaggaacccagcactccactggataagcattatccttatccaaaacagccttgtggtcagtgttcatctgctgactgtcaactgtagcattttttggggttacagtttgagcaggatatttggtcctgtagtttgctaacacaccctgcagctccaaaggttccccaccaacagcaaaaaaatgaaaatttgacccttgaatgggttttccagcaccattttcatgagttttttgtgtccctgaatgcaagtttaacatagcagttaccccaataacctcagttttaacagtaacagcttcccacatcaaaatatttccacaggttaagtcctcatttgtagaattcgccagcacagtggtcgaccctgtggatgtgtgtcacttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaagcttaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagaacttgtttattgcagcttataatggttacaaataaagcaatagcatcacaaatttcacaaataaagcatttttttcactgcattctagttgtggtttgtccaaactcatcaatgtatcttatcatgtctggatcgatcccgccatggtatcaacgccatatttctatttacagtagggacctcttcgttgtgtaggtaccccgggttcgaaatcgaattcgccaatgacaagacgctgggcggggtttgtgtcatcatagaactaaagacatgcaaatatatttcttccggggacaccgccagcaaacgcgagcaacgggccacggggatgaagcagcccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcggggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcgccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcgtagacatcatgcgtgctgttggtgtatttctggccatctgtcttgtcaccattttcgtcctcccaacatggggcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgcccc

SEQ ID NO: 35 - protein, 98 aa, human; identical to SEQ ID NO: 13

SEQ ID NO: 36 - protein, 12 aa, human
ERKCCVECPPCP

SEQ ID NO: 37 - protein, 107 aa, human; identical to SEQ ID NO: 11

SEQ ID NO: 38 - DNA, 11192 bp, artificial sequence
  vector
gtgaccaatacaaaacaaaagcgcccctcgtaccagcgaagaaggggcagagatgccgtagtcaggtttagttcgtccggcggcgggggatctgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagaattctgttgggctcgcggttgattacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgaccggcttggtaccgagctcggatccgcggccgcgtacggccgccaccatggattgggtctggaccttgctattcctcttgtcagtaactgcaggtgtccactcacaggtccagctgcaacagtctggggctgaactcgcaagacctggggcctcagtgaagatgtcctgcaaggcttctggctacacgtttactaggtacacgatgcactgggtaaaacagaggcctggacaaggtttggaatggattggatacattaaccctagccgtggatatactaattacaatcagaagttcaaggacaaggccacactgactacagacaaatcttccagcacagcctacatgcaactgagcagcctgacatctgaggactccgcagtctattactgtgcaagatattatgatgatcattactgtctcgactactggggccaaggcaccactttgacagtctcctcaggtgagtccttacaacctctctcttctattcagcttaaatagattttactgcatttgttgggggggaaatgtgtgtatctgaatttcaggtcatgaaggactagggacaccttgggagtcagaaagggtcattgggagcccgggctgatgcagacagacatcctcagctcccagacttcatggccagagatttataggatcctctagattgagctttctggggcaggccaggcctgaccttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcctccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccaggcaggcacaggctgggtgcccctaccccaggcccttcacacacaggggcaggtgcttggctcagacctgccaaaagccatatccgggaggaccctgcccctgacctaagccgaccccaaaggccaaactgtccactccctcagctcggacaccttctctcctcccagatccgagtaactcccaatcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgaggaattcagatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccccaggaagctcctctgtgtcctcataaaccctaacctcctctacttgagaggacattccaatcataggctgcccatccaccctctgtgtcctcctgttaattaggtcacttaacaaaaaggaaattgggtaggggtttttcacagaccgctttctaagggtaattttaaaatatctgggaagtcccttccactgctgtgttccagaagtgttggtaaacagcccacaaatgtcaacagcagaaacatacaagctgtcagctttgcacaagggcccaacaccctgctcatcaagaagcactgtggttgctgtgttagtaatgtgcaaaacaggaggcacattttccccacctgtgtaggttccaaaatatctagtgttttcatttttacttggatcaggaacccagcactccactggataagcattatccttatccaaaacagccttgtggtcagtgttcatctgctgactgtcaactgtagcattttttggggttacagtttgagcaggatatttggtcctgtagtttgctaacacaccctgcagctccaaaggttccccaccaacagcaaaaaaatgaaaatttgacccttgaatgggttttccagcaccattttcatgagttttttgtgtccctgaatgcaagtttaacatagcagttaccccaataacctcagttttaacagtaacagcttcccacatcaaaatatttccacaggttaagtcctcatttgtagaattcgccagcacagtggtcgaccctgtggatgtgtgtcacttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaagcttaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagaacttgtttattgcagcttataatggttacaaataaagcaatagcatcacaaatttcacaaataaagcatttttttcactgcattctagttgtggtttgtccaaactcatcaatgtatcttatcatgtctggatcgatcccgccatggtatcaacgccatatttctatttacagtagggacctcttcgttgtgtaggtaccccgggttcgaaatcgaattcgccaatgacaagacgctgggcggggtttgtgtcatcatagaactaaagacatgcaaatatatttcttccggggacaccgccagcaaacgcgagcaacgggccacggggatgaagcagcccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcggggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcgccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcgtagacatcatgcgtgctgttggtgtatttctggccatctgtcttgtcaccattttcgtcctcccaacatggggcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgcccc

SEQ ID NO: 39 - protein, 138 aa, murine; identical to SEQ ID NO: 29

SEQ ID NO: 40 - protein, 98 aa, human; identical to SEQ ID NO: 13

SEQ ID NO: 41 - protein, 12 aa, human
ERKCCVECPPCP

SEQ ID NO: 42 - protein, 107 aa, human
APPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISK

SEQ ID NO: 43 - protein, 107 aa, human; identical to SEQ ID NO: 11

SEQ ID NO: 44 - DNA, 6099 bp, artificial sequence
  vector
aagcttatgaatatgcaaatcctctgaatctacatggtaaatataggtttgtctataccacaaacagaaaaacatgagatcacagttctctctacagttactgagccgtacggccgccaccatggattttcaagttcagattttcagcttcctgctaatcagtgcctcagtcataatatccagaggacaaattgttctcacccagtctccagcaatcatgtctgcatctccaggggaaaaggtcaccatgacatgcagtgccagctcaagtgtaagttacatgaactggtaccagcagaagtcaggcacctcccccaaaagatggatttatgacacatcaaaactggcttctggagtaccggctcacttcaggggcagtgggtctgggacctcttactctctcacaatctcagggatggaagctgaagatgccgcaacttattactgccagcagtggtcaagtaacccattcacgttcggatctggtacaaagttggaaatcaaacgtgagtagactgcacactttgcttcgtcgactggatcctggcagagtctcacagatgcttctgagacaacatttgctttcaaaaaatgaaccacacacatcctaaagatctcagccacttcccatgtttcattttatgttacagcaaacatcacaacaatcattcctacagatcaccactgcatgtgatcaataaaatagtttttgcaacaatggtacttatgataatcatcttttattgtttacaaatactgctttacaatagttattcggttgcactgttcatattagatttccaattagctcacttaggaacataagtccctcgaacagctcagtcatctttttcattcctgtttctatcccctacatctctttcctttgcagacgactatctcctacactgaaacaggaaagctagcttttttttttcagtgctatttaattatttcaatatcctctcatcaaatgtatttaaataacaaaagctcaaccaaaaagaaagaaatatgtaattctttcagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgataggactattttagggcctttttaaagctctattaaaactaacttacaacgattcaaaactgttttaaactatttcaaaatgattttagagccttttgaaaactcttttaaacactttttaaactctattaaaactaataagataacttgaaataattttcatgtcaaatacattaactgtttaatgtttaaatgccagatgaaaaatgtaaagctatcaagaattcacccagataggagtatcttcatagcatgtttttccctgcttattttccagtgatcacattattttgctaccatggttattttatacaattatctgaaaaaaattagttatgaagattaaaagagaagaaaatattaaacataagagattcagtctttcatgttgaactgcttggttaacagtgaagttagttttaaaaaaaaaaaaaactatttctgttatcagctgacttctccctatctgttgacttctcccagcaaaagattcttattttacattttaactactgctctcccacccaacgggtggaatcccccagagggggatttccaagaggccacctggcagttgctgagggtcagaagtgaagctagccacttcctcttaggcaggtggccaagattacagttgacctctcctggtatggctgaaaattgctgcatatggttacaggccttgaggcctttgggagggcttagagagttgctggaacagtcagaaggtggaggggctgacaccacccaggcgcagaggcagggctcagggcctgctctgcagggaggttttagcccagcccagccaaagtaacccccgggagcctgttatcccagcacagtcctggaagaggcacaggggaaataaaagcggacggaggctttccttgactcagccgctgcctggtcttcttcagacctgttctgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgttttaccaactactcaatttctcttataagggactaaatatgtagtcatcctaaggcgcataaccatttataaaaatcatccttcattctattttaccctatcatcctctgcaagacagtcctccctcaaacccacaagccttctgtcctcacagtcccctgggccatggtaggagagacttgcttccttgttttcccctcctcagcaagccctcatagtcctttttaagggtgacaggtcttacagtcatatatcctttgattcaattccctgagaatcaaccaaagcaaatttttcaaaagaagaaacctgctataaagagaatcattcattgcaacatgatataaaataacaacacaataaaagcaattaaataaacaaacaatagggaaatgtttaagttcatcatggtacttagacttaatggaatgtcatgccttatttacatttttaaacaggtactgagggactcctgtctgccaagggccgtattgagtactttccacaacctaatttaatccacactatactgtgagattaaaaacattcattaaaatgttgcaaaggttctataaagctgagagacaaatatattctataactcagcaatcccacttctagatgactgagtgtccccacccaccaaaaaactatgcaagaatgttcaaagcagctttatttacaaaagccaaaaattggaaatagcccgattgtccaacaatagaatgagttattaaactgtggtatgtttatacattagaatacccaatgaggagaattaacaagctacaactatacctactcacacagatgaatctcataaaaataatgttacataagagaaactcaatgcaaaagatatgttctgtatgttttcatccatataaagttcaaaaccaggtaaaaataaagttagaaatttggatggaaattactcttagctgggggtgggcgagttagtgcctgggagaagacaagaaggggcttctggggtcttggtaatgttctgttcctcgtgtggggttgtgcagttatgatctgtgcactgttctgtatacacattatgcttcaaaataacttcacataaagaacatcttatacccagttaatagatagaagaggaataagtaataggtcaagaccatgcagctggtaagtgggggggcctgggatcaaatagctacctgcctaatcctgccctcttgagccctgaatgagtctgccttccagggctcaaggtgctcaacaaaacaacaggcctgctattttcctggcatctgtgccctgtttggctagctaggagcacacatacatagaaattaaatgaaacagaccttcagcaaggggacagaggacagaattaaccttgcccagacactggaaacccatgtatgaacactcacatgtttgggaagggggaagggcacatgtaaatgaggactcttcctcattctatggggcactctggccctgcccctctcagctactcatccatccaacacacctttctaagtacctctctctgcctacactctgaaggggttcaggagtaactaacacagcatcccttccctcaaatgactgaccatccctttgtcctgctttgtttttctttccagtcagtactgggaaagtggggaaggacagtcatggaaaaactacataaggaagcaccttgcccttctgcctcttgagaatgttgatgagtatcaaatctttcaaactttggaggtttgagtaggggtgagactcagtaatgtcccttccaatgacatgaacttgctcactcatccctgggggccaaattgaacaatcaaaggcaggcataatccagttatgaattcaaaccttcttctcagaagataacactctgaagggaaacccacccataacctaagcaagtgaagacaggtgctgcaggtggaattgtgtccttcaaaaaggtatgctcaactccttgctcttggtactcataaatgggtcacataaatgtgactttatttggaaatagggtctttgcagaggtaatcaagtcaaaattaggtcatactgaaatgtttgtgaggatgcggtgaaaatggatcattcatatattgctggtgggaatataaaagggtatagctactctagaaaatagttgtcagtttcttgaaaaactaaacaaaagacacctaccatatgacccaggaattgtactccttgggaatttacccccaggaaataaaaacttatgtccacacagaacccatacatgattgttcacagcagctttatttgttgtagccaaagctagaaagagccaacccatccctcaataggcaactagcctaacaaattgtaatatatccatgccatagaatgctatgaggcaataaaaaggaacgaagtgttgatacagagaactggagtgattctgaaggactttctactgagtgaaaaaagccaatctgaaagggtcacataccatgtgattccttttatgtaacattgttgaagtgacaaaattatagggatagagaacagattctggttgccaggggttagggtggtggagaaagaagagtaggcgaaactataaagggagatctttgtgatcatgggataaatctgtatcttgattgcagtggtagttgcaggcatctagacatgtgataaaatgacatagaactgtacacacttattttatcaatgtcaaattcttggttttaatatcgtactgtaattacgtaagaagtaaccaacaggagaaactgggtgcaggacacatcagacctctgtgctttatatcctgtctttgctactttctgtgaatctataattatttccaaataatttttttaaactttttttttatgctggatcg

SEQ ID NO: 45 - protein, 129 aa, murine
MDFQVQIFSFLLISASVIISRGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKR

SEQ ID NO: 46 - DNA, 3535 bp, artificial sequence
  shuttle vector
gttaactacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttctccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtaatgtgagttagctcactcattaggcaccccaggctttacactttatgcttccggctcgtatgttgtgtggaattgtgagcggataacaatttcacacaggaaacagctatgaccatgattacgccaagctacgtaatacgactcactagtgggcagatcttcgaatgcatcgcgcgcaccgcgtacggccgccaccatggattttcaagttcagattttcagcttcctgctaatcagtgcctcagtcataatatccagaggacaaattgttctcacccagtctccagcaatcatgtctgcatctccaggggaaaaggtcaccatgacatgcagtgccagctcaagtgtaagttacatgaactggtaccagcagaagtcaggcacctcccccaaaagatggatttatgacacatcaaaactggcttctggagtaccggctcacttcaggggcagtgggtctgggacctcttactctctcacaatctcagggatggaagctgaagatgccgcaacttattactgccagcagtggtcaagtaacccattcacgttcggatctggtacaaagttggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcctgcaggatatctggatccacgaagcttcccatggtgacgtcaccggttctagatacctaggtgagctctggtaccctctagtcaaggccttaagtgagtcgtattacggactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcttcgcttggtaataaagcccgcttcggcgggctttttttt

SEQ ID NO: 47 - protein, 235 aa, murine
MDFQVQIFSFLLISASVIISRGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - DNA, 9701 bp, artificial sequence
  vector
gtgaccaatacaaaacaaaagcgcccctcgtaccagcgaagaaggggcagagatgccgtagtcaggtttagttcgtccggcggcgggggatctgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagaattctgttgggctcgcggttgattacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgaccggcttggtaccgagctcggatcttcgaatgcatcgcgcgcaccgcgtacggccgccaccatggattttcaagttcagattttcagcttcctgctaatcagtgcctcagtcataatatccagaggacaaattgttctcacccagtctccagcaatcatgtctgcatctccaggggaaaaggtcaccatgacatgcagtgccagctcaagtgtaagttacatgaactggtaccagcagaagtcaggcacctcccccaaaagatggatttatgacacatcaaaactggcttctggagtaccggctcacttcaggggcagtgggtctgggacctcttactctctcacaatctcagggatggaagctgaagatgccgcaacttattactgccagcagtggtcaagtaacccattcacgttcggatctggtacaaagttggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggaattcctgcaggatatctgggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccccaggaagctcctctgtgtcctcataaaccctaacctcctctacttgagaggacattccaatcataggctgcccatccaccctctgtgtcctcctgttaattaggtcacttaacaaaaaggaaattgggtaggggtttttcacagaccgctttctaagggtaattttaaaatatctgggaagtcccttccactgctgtgttccagaagtgttggtaaacagcccacaaatgtcaacagcagaaacatacaagctgtcagctttgcacaagggcccaacaccctgctcatcaagaagcactgtggttgctgtgttagtaatgtgcaaaacaggaggcacattttccccacctgtgtaggttccaaaatatctagtgttttcatttttacttggatcaggaacccagcactccactggataagcattatccttatccaaaacagccttgtggtcagtgttcatctgctgactgtcaactgtagcattttttggggttacagtttgagcaggatatttggtcctgtagtttgctaacacaccctgcagctccaaaggttccccaccaacagcaaaaaaatgaaaatttgacccttgaatgggttttccagcaccattttcatgagttttttgtgtccctgaatgcaagtttaacatagcagttaccccaataacctcagttttaacagtaacagcttcccacatcaaaatatttccacaggttaagtcctcatttaaattaggcaaaggaattcctcgacctgcagcccaagcttggcactggcgccagaaatccgcgcggtggtttttgggggtcgggggtgtttggcagccacagacgcccggtgttcgtgtcgcgccagtacatgcggtccatgcccaggccatccaaaaaccatgggtctgtctgctcagtccagtcgtggacctgaccccacgcaacgcccaaaagaataacccccacgaaccataaaccattccccatgggggaccccgtccctaacccacggggcccgtggctatggcgggcttgccgcccccacgttggctgcgagccctgggccttcacccgaacttgggggttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggattgtctccttccgtgtttcagttagcctcccccatctcccgatccccacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatcaagctgatcttgcggcacgctgttgacgctgttaagcgggtcgctgcagggtcgctcggtgttcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagcccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcggggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcgccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcgtagacatcatgcgtgctgttggtgtatttctggccatctgtcttgtcaccattttcgtcctcccaacatggggcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgcccc

SEQ ID NO: 49 - protein, 235 aa, artificial sequence; identical to SEQ ID NO: 47
  murine/human fusion

SEQ ID NO: 50 - DNA, 13 bp, artificial sequence
  adaptor duplex
gatccgcggccgc

SEQ ID NO: 51 - DNA, 13 bp, artificial sequence
  adaptor duplex
gcgccggcgcatg

SEQ ID NO: 52 - DNA, 36 bp, artificial sequence
  oligonucleotide
cagatgtgatatctccaaggtccccagctccctaag

SEQ ID NO: 53 - DNA, 32 bp, artificial sequence
  oligonucleotide
tgggctcgagttcgtctctgaagcaggctgcg

SEQ ID NO: 54 - DNA, 32 bp, artificial sequence
  primer
agacgaactcgagcgcaaatgttgtgtcgagt

SEQ ID NO: 55 - DNA, 39 bp, artificial sequence
  primer
cggccctggcactcatttacccagagacagggagaggct

SEQ ID NO: 56 - DNA, 78 bp, artificial sequence
  leader sequence
cgcccttccaccatggacatgagggtccccgctcagctcctggggctcctgctactctggctccgaggtgccagatgt

SEQ ID NO: 57 - protein, 98 aa, human
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 58 - protein, 107 aa, human; identical to SEQ ID NO: 42

SEQ ID NO: 59 - protein, 137 aa, murine; identical to SEQ ID NO: 8

SEQ ID NO: 60 - protein, 106 aa, human
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2007/0041972 A1
(43) Pub. Date: Feb. 22, 2007

(54) Antibodies and fusion proteins that include engineered constant regions

(75) Inventors: Russell Rother, Prospect, CT (US); Dayang Wu, Cheshire, CT (US); Stephen P. Squinto, Bethany, CT (US); Mark Evans, Radnor, PA (US); Susan Faas McKnight, Old Lyme, CT (US)

Correspondence Address: FISH & NEAVE IP GROUP;ROPES & GRAY LLP, ONE INTERNATIONAL PLACE, BOSTON, MA, 02110-2624, US

(73) Assignee: ALEXION PHARMACEUTICALS, INC., CHESHIRE, CT (US)

(21) Appl. No.: 10/559,085

(22) PCT Filed: May 28, 2004
(86) PCT No.: PCT/US04/17005

§ 371 Date: Jul. 24, 2006

Related U.S. Application Data
(60) Provisional application No. 60/475,202, filed on May 30, 2003.
(60) Provisional application No. 60/563,500, filed on Apr. 19, 2004.

(51) Int. Cl. A61K 39/395; C07H 21/04; C12P 21/06; C07K 16/24; C07K 16/46; C12N 5/06

(52) U.S. Cl. 424/145.1; 424/178.1; 530/388.23; 530/391.1; 435/69.1; 435/326; 435/320.1; 536/23.53

(57) ABSTRACT

Antibodies and/or fusion proteins contain a region that includes an IgG2-derived portion IgG4-derived portion.

### RELATED CASES

This application claims priority to U.S. Provisional Applications 60/475,202 and 60/563,500 filed May 30, 2003 and Apr. 19, 2004, respectively, the entire disclosures of which are incorporated herein by this reference.

### BACKGROUND

1. Technical Field

The present disclosure relates to the field of genetically engineered antibodies and fusion proteins. More specifically this disclosure relates to antibodies and/or fusion proteins containing a region that includes an IgG2-derived portion and an IgG4-derived portion.

2. Background of Related Art

Antibodies are produced by B lymphocytes and defend against infections. The basic structure of an antibody consists of two identical light polypeptide chains and two identical heavy polypeptide chains linked together by disulphide bonds. The first domain located at the amino terminus of each chain is variable in amino acid sequence, providing the vast spectrum of antibody binding specificities found in each individual. These are known as variable heavy (VH) and variable light (VL) regions. The other domains of each chain are relatively invariant in amino acid sequence and are known as constant heavy (CH) and constant light (CL) regions. The major classes of antibodies are IgA, IgD, IgE, IgG and IgM; and these classes may be further divided into subclasses (isotypes). For example, the IgG class has four subclasses, namely, IgG1, IgG2, IgG3, and IgG4. Of the various human antibody classes, only human IgG1, IgG2, IgG3 and IgM are known to effectively activate the complement system.

The differences between antibody classes are derived from differences in the heavy chains. Class switching is known to occur during antibody maturation. The basic antibody molecule is a bifunctional structure wherein the variable regions bind antigen while the remaining constant regions elicit certain effector functions. The hinge region is particularly sensitive to proteolytic cleavage; such proteolysis yields two or three fragments depending on the precise site of cleavage. The hinge region allows the antigen binding regions (each made up of a light chain and the first two domains of a heavy chain) to move or rotate freely relative to the rest of the antibody, which includes the remaining heavy chain domains. Although the constant regions do not form the antigen binding sites, the arrangement of the constant domains and hinge region confer segmental flexibility on the molecule which allows it to bind with the antigen.

The interaction between the antigen and the antibody takes place by the formation of multiple bonds and attractive forces such as hydrogen bonds, electrostatic forces and Van der Waals forces. Together these form considerable binding energy which allows the antibody to bind the antigen. Antibody binding affinity and avidity have been found to affect the physiological and pathological properties of antibodies.

The advent of genetic engineering technology has led to various means of producing unlimited quantities of uniform antibodies (monoclonal antibodies) which, depending upon the isotype, exhibit varying degrees of effector function. For example, certain murine isotypes (IgG1, IgG2) as well as human isotypes (particularly IgG1) can bind to Fc receptors on cells such as monocytes, B cells and NK cells, thereby activating the cells to release cytokines; such antibody isotypes are also potent in activating complement, with local or systemic inflammatory consequences. When antibodies bearing these Fc receptor-binding constant regions are injected in vivo, a transient but significant systemic release of tumor necrosis factor alpha (TNF-α), interferon gamma (IFN-γ), interleukin 2 (IL-2) and/or other cytokines may be released as a consequence of activation of multiple cell types including lymphocytes or monocytes through Fc receptor-antibody engagement. The release of cytokines is usually accompanied by high fever, chills and headache, but less frequently may progress to more severe and potentially life-threatening symptoms, such as pulmonary edema, meningitis, neurotoxicity, hypotension and respiratory distress (cytokine release syndrome or CRS). The murine antibody OKT3 is one antibody that has been observed to cause significant cytokine release leading to CRS. The human CD3 moiety consists of at least four invariant polypeptide chains, which are non-covalently associated with the T cell receptors (TCR) on the surface of T-cells, typically referred to as the T cell receptor complex. The T cell receptor complex plays an important role in the T-cell activation upon antigen binding to the T cell receptor. Some anti-CD3 antibodies, such as OKT3, can activate T-cells in the absence of antigen-TCR ligation. Such activation depends upon the interaction between the Fc portion of the mAb and the Fc receptors on accessory cells, enabling crosslinking of CD3 complexes on the T-cells. Soluble anti-CD3 mAbs do not stimulate T-cells to proliferate in vitro unless they are bound to plastic (which artificially promotes CD3 cross-linking) or bound to Fc receptor-bearing cells.

It would be desirable to reduce antibody-mediated cell activation events such as, for example, cytokine release in settings where these events are not warranted and/or harmful. Numerous laboratories have attempted to reduce the negative effects associated with potent effector function, such as that observed with OKT3, by engineering antibodies with different constant regions that exhibit features such as reduced Fc receptor binding, lack of complement activation, etc.

Therefore it is an object herein to reduce effector function in engineered antibodies through the incorporation of unique constant regions.

### SUMMARY

Recombinant antibodies having engineered heavy chain constant regions are described herein. The engineered constant regions include an IgG2-derived portion and an IgG4-derived portion. Preferably, the IgG2-derived portion includes at least the heavy chain constant region 1 and hinge region and the IgG4-derived portion includes most of the heavy chain constant region 2 and the entire heavy chain constant region 3. Antibodies that bind cell surface molecules or soluble molecules that bind to cell surface molecules having an engineered heavy chain constant region in accordance with this disclosure, reduce unwanted antibody-mediated cell activation and inflammatory events (including reduced complement activation) resulting from Fc-receptor antibody engagement.

In another aspect, this disclosure relates to a process for producing an antibody heavy chain which includes the steps of: (a) producing an expression vector having a DNA sequence which includes a sequence that encodes an antibody heavy chain containing a variable region and a constant region; (b) having said constant region comprised of a first portion derived from one or more human IgG2 antibodies and a second portion derived from one or more human IgG4 antibodies; (c) transfecting a host cell with the vector; and (d) culturing the transfected cell line to produce an engineered antibody heavy chain molecule which associates with antibody light chains to produce a functional antibody molecule.

In another aspect, this disclosure relates to methods for reducing antibody-mediated cell activation and inflammatory events through binding cell surface molecules or soluble molecules that bind to cell surface molecules using antibodies having an engineered heavy chain constant region having a first portion derived from one or more human IgG2 antibodies and a second portion derived from one or more human IgG4 antibodies, associated with light chains to produce a functional antibody molecule.

In another embodiment, an engineered constant region that includes an IgG2-derived portion and an IgG4-derived portion in accordance with this disclosure is used as the Fc region of a fusion protein. The fusion proteins include a non-Fc component fused to an Fc region that is engineered to include an IgG2-derived portion and an IgG4-derived portion. Preferably, the IgG2-derived portion includes at least the heavy chain constant region 1 and hinge region and the IgG4-derived portion includes most of the heavy chain constant region 2 and the entire heavy chain constant region 3. Preferably the fusion proteins in accordance with this disclosure maintain the function of the non-Fc component and/or have increased half-life compared to the non-Fc component alone and/or lack unwanted antibody Fc-mediated cell activating and inflammatory properties including events resulting from Fc-receptor antibody engagement and complement activation.

Recombinant DNA molecules encoding such fusion proteins are also provided. Upon heterologous expression in transfected mammalian cells, the fusion proteins are potently secreted in stable form, and display desired properties characteristic of the antibody and non-Fc component predecessor molecules. These fusion proteins can be used in applications conventionally associated with monoclonal antibodies, including flow cytometry, immunohisto-chemistry, cell-based assays and immunoprecipitation.

### BRIEF DESCRIPTION OF THE DRAWINGS

FIGS. 1A, B, and C schematically shows examples of a chimeric (FIG. 1A), humanized (FIG. 1B) or fully human (FIG. 1C) recombinant antibody, respectively, having an engineered heavy chain constant region in accordance with this disclosure.

FIG. 2 shows the amino acid sequence (SEQ ID NO: 1) of an engineered heavy chain constant region in accordance with this disclosure and the nucleotide sequence (SEQ ID NO: 2) that encodes that engineered heavy chain constant region.

FIGS. 3A and 3B show the human IgG2 (GenBank Accession number V00554), and human IgG4 (GenBank Accession number K01316) amino acid and nucleic acid sequences, respectively. FIG. 3C shows a schematic map of the plasmid pBR322 (GenBank Accession number J01749).

FIG. 4A shows a graphic map of the vector APEX-1 3F4V_{H}HuGamma4. FIG. 4B shows the complete nucleotide sequence of the vector (SEQ ID NO: 3) and indicates the amino acid (SEQ ID NO: 4) and nucleotide sequences of the hIgG4 insert adjacent to an irrelevant VH region (labeled 3F4VH). The locations of the signal sequence, CH1, hinge, CH2 and CH3 regions are indicated.

FIG. 5A shows a graphic map of the vector APEX-1 3F4V_{H}HuG2/G4. FIG. 5B shows the nucleotide sequence of the vector (SEQ ID NO: 5) and the amino acid (SEQ ID NO: 6) and nucleic acid sequence of the G2/G4 insert, and indicates the locations of the signal sequence, irrelevant Vh (herein labeled 3F4Vh), CH1, hinge, CH2 and CH3 regions.

FIG. 6 shows the complete nucleotide and amino acid sequence of the OKT3 heavy chain variable region (GenBank Accession number A22261).

FIG. 7 shows the complete nucleotide and amino acid sequence of the OKT3 light chain variable region (GenBank Accession number A22259).

FIG. 8 shows the complete nucleotide (SEQ ID NO: 7) and amino acid (SEQ ID NO:8) sequences of the murine OKT3 heavy chain variable region, constructed using Expression Strategy #1, and including the murine immunoglobulin promoter, a murine signal sequence with intron at the 5′ ends, and a splice donor site (Bam HI) at the 3′ ends. Restriction enzyme sites are indicated.

FIG. 9 shows the complete nucleotide (SEQ ID NO: 9) and amino acid (SEQ ID NO:10) sequences of the murine OKT3 light chain variable region, constructed using Expression Strategy #1, and including the murine immunoglobulin promoter, a murine signal sequence with intron at the 5′ ends, and a splice donor site (Bam HI) at the 3′ ends. Restriction enzyme sites are indicated.

FIG. 10 shows the complete nucleotide sequence (SEQ ID NO: 11) of the HuG2/G4 fragment excised from the APEX-1 3F4V_{H}HuG2/G4 vector and modified for insertion into a PUC 19 cloning vector by the addition, at the 5′ end, of a Bam HI site and 5′ untranslated inron sequences from native human IgG4 and, at the 3′ end, of a Bgl II site and 3′ untranslated sequence from natural human IgG4.

FIG. 11 shows a graphic map of the heavy chain expression vector pSVgptHuG2/G4 used in Expression System #1.

FIG. 12 shows a graphic map of the expression vector pSVgptHuCk used in Expression System #1.

FIGS. 13A, B, and C show the nucleotide (SEQ ID NO: 12) and amino acid (SEQ ID NO: 13) sequences of the OKT3 heavy chain variable region and huG2/G4 constant region constructed using Expression Strategy #2. The construct lacks the 5′ leader intron and employs the original OKT3 signal sequence (indicated). Restriction enzyme sites are also indicated.

FIGS. 14A-D show the entire nucleic acid sequence of the APEX-3P G2/G4 expression vector used in Expression System #2 (SEQ ID NO: 14), including the amino acid sequence (SEQ ID NO: 15) of the G2/G4 insert and indicated restriction sites.

FIGS. 15A and B show the entire nucleic acid sequence of the expression vector APEX-3PmOKT3VhG2G4 (SEQ ID NO: 16), used in Expression System #2, including the amino acid sequence (SEQ ID NO: 17) of the OKT3 variable heavy region and G2/G4 insert. Restriction enzyme sites are indicated.

FIGS. 16A-F show the entire nucleic acid sequence of PUC19 (SEQ ID NO: 18) and the amino acid sequence (SEQ ID NO: 19) of the OKT3Vk and human Ck expression cassettes used in Expression System #2. Restriction enzyme sites are indicated.

FIG. 17 shows the entire nucleic acid sequence of the shuttle vector, LITMUS 28 (SEQ ID NO: 20), and the amino acid sequences of the OKT3VkhCk insert (SEQ ID NO: 21) used in Expression System #2.

FIGS. 18A and B show the entire nucleic acid sequence of APEX-3 OKT3Vk+Ck (SEQ ID NO: 22) and the amino acid sequences of the OKT3Vk and Ck insert (SEQ ID NO: 23) used in Expression System #2. Restriction enzyme sites are indicated.

FIGS. 19A and B show the results of tests evaluating the ability of an antibody containing the G2/G4 heavy chain constant region to bind to the FcγRI receptor on U937 cells.

FIG. 20 shows the results of tests evaluating the ability of an antibody containing the G2/G4 heavy chain constant region to bind to the FcγRII receptor on K562 cells.

FIG. 21 shows the results of tests designed to evaluate the ability of an antibody containing the G2/G4 heavy chain constant region to induce cytokine production in human PBL.

FIG. 22 shows the results of tests designed to evaluate the ability of an antibody containing the G2/G4 heavy chain constant region to induce the upregulation of activation markers on human T cells.

### DETAILED DESCRIPTION OF THE PREFERRED EMBODIMENTS

Recombinant antibodies are described with engineered heavy chain constant regions that serve to reduce antibody-mediated cell activation and inflammation events resulting from antibody-Fc receptor interactions. The engineered heavy chain constant region includes a portion derived from one or more human antibodies of the IgG2 sub-class and a portion derived from one or more human antibodies of the IgG4 sub-class. As those skilled in the art will appreciate, an antibody heavy chain includes a variable region and a constant region. The heavy chain constant region includes the heavy chain constant region 1 (CH1), hinge region, heavy chain constant region 2 (CH2) and heavy chain constant region 3 (CH3). At least a portion of one of the CH1, hinge region, CH2 or CH3 are derived from a human IgG2 antibody in the present engineered heavy chains, with at least a portion of the balance of the engineered heavy chain being derived from a human IgG4 antibody. Preferably, the entire engineered heavy chain is derived from a combination of human IgG2 and IgG4 portions.

It should be understood that two or more, non-contiguous portions of the heavy chain constant region can be derived from an IgG2 antibody. In such circumstances, the portions can be derived from the same or from different antibodies (i.e. those with different allotypes) within the IgG2 subclass. Likewise, two or more, non-contiguous portions of the heavy chain constant region can be derived from an IgG4 antibody (note that there is only one known IgG4 allotype).

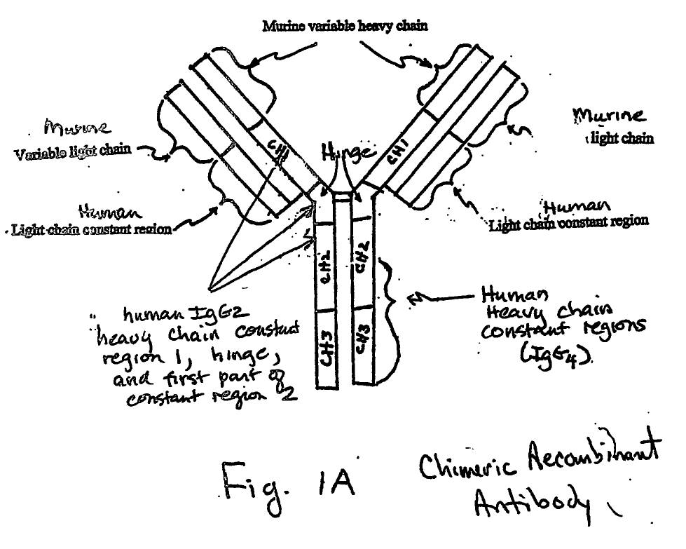

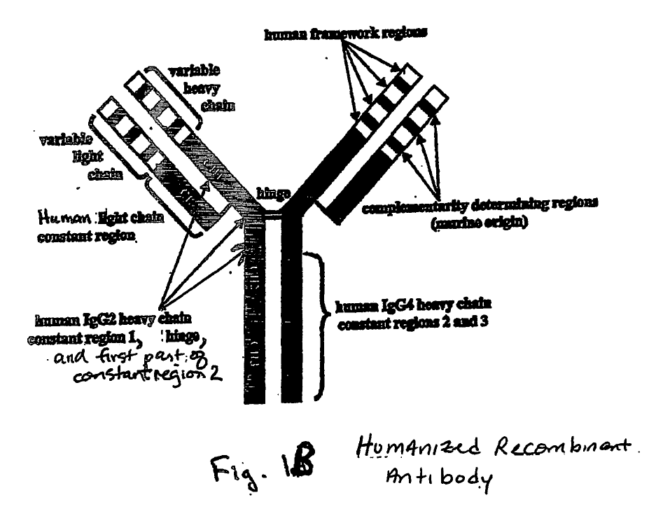

In a particularly useful embodiment shown schematically in FIG. 1, the engineered heavy chain constant region includes a CH1 and hinge region derived from one or more human antibodies of the IgG2 sub-class and CH2 and CH3 regions derived primarily from an antibody of the IgG4 sub-class. The engineered antibody can be in the form of a mouse-human chimeric antibody (FIG. 1A); a humanized antibody (FIG. 1B) or a fully human antibody (FIG. 1C).

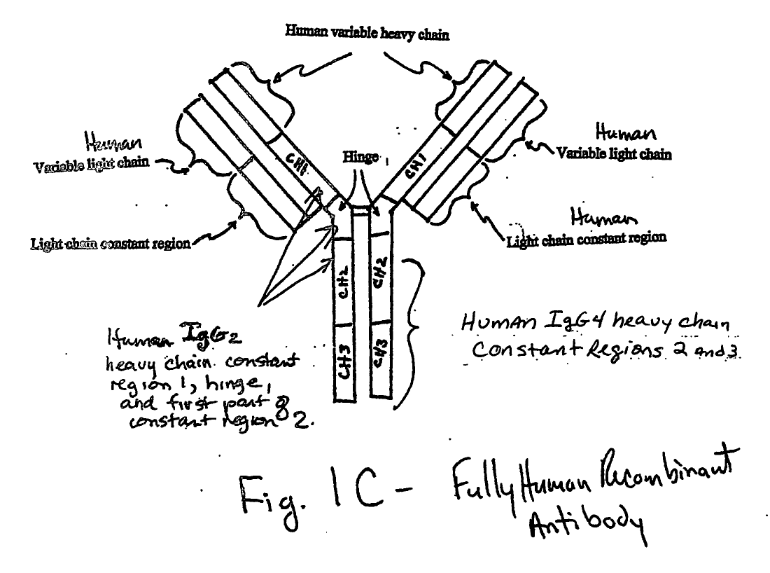

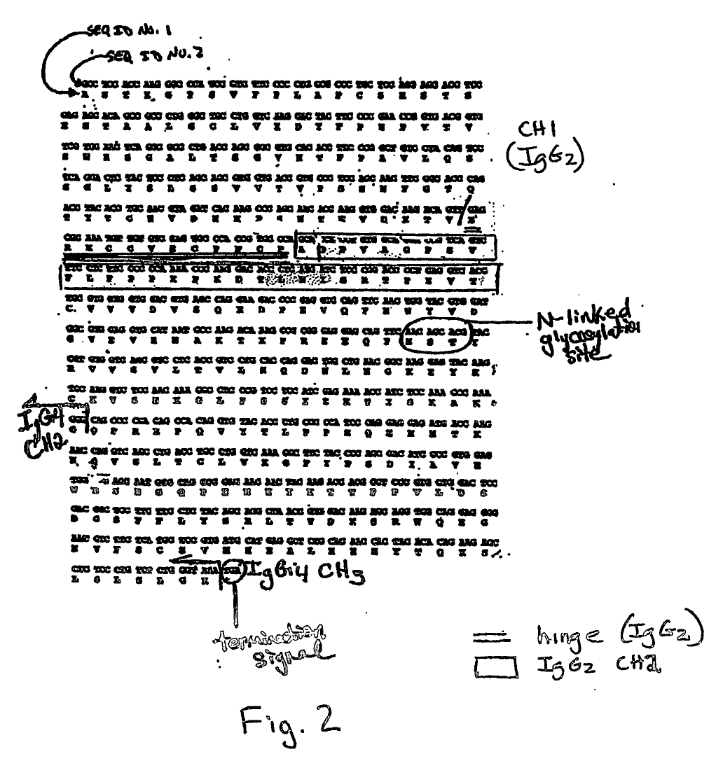

It should be understood that the portion derived from an IgG2 antibody and the portion derived from an IgG4 antibody need not terminate precisely at the junction between constant regions and/or the hinge region. For instance, in the working examples presented below (see FIG. 2), the portion derived from an IgG2 antibody extends beyond the hinge region by a few amino acids into the constant region 2, with the balance of the heavy chain constant region being the portion derived from an IgG4 antibody.

One example of an engineered heavy chain constant region in accordance with this disclosure has the sequence shown in FIG. 2 (SEQ ID NO: 1). FIG. 2 also shows the nucleotide sequence (SEQ ID NO: 2) that encodes the engineered heavy chain constant region.

The IgG2 and IgG4 portions of the heavy chain constant region are chosen to reduce cell interactions that can result in excessive cytokine release, potentially leading to cytokine release syndrome (CRS). The engineered heavy chain constant region in accordance with the present disclosure also reduces the ability of the antibody to elicit inflammatory events such as cell activation, cytokine release and complement activation.

The heavy chain variable region of the antibody is selected for its binding specificity and can be of any type, such as, for example, non-human, humanized or fully human. Where the heavy chain variable region of the antibody is non-human (such as, for example, murine) and is combined recombinantly with an engineered heavy chain constant region in accordance with this disclosure, the resulting recombinant antibody is referred to as a chimeric antibody (see FIG. 1A). Where the heavy chain variable region of the antibody is humanized and is combined recombinantly with an engineered heavy chain constant region in accordance with this disclosure, the resulting recombinant antibody is referred to as a humanized antibody (see FIG. 1B). Where the heavy chain variable region of the antibody is human and is combined recombinantly with an engineered heavy chain constant region in accordance with this disclosure, the resulting recombinant antibody is referred to as a fully human antibody (see FIG. 1C). In the embodiment shown in FIG. 1B, the variable region of the heavy chain is humanized and includes human framework regions and non-human (in this case murine) complementary determining regions (CDRs). It should be understood that the framework regions can be derived from one source or more than one source and that the CDRs can be derived from one source or more than one source. Methods for humanization of antibodies are known to those skilled in the art and are disclosed, for example, in U.S. Pat. Nos. 6,479,284; 6,407,213; 6,350,861; 6,180,370; 6,548,640; and commonly owned, pending U.S. Patent Application Serial No. PCT/US 02/ 38450, Filed Dec. 3, 2002. The disclosures of each of these patents and patent applications are incorporated herein in their entirety by this reference.

The light chain of the antibody can be human, non-human or humanized. In the embodiment shown in FIG. 1B, the light chain is humanized and includes human framework regions, non-human (in this case murine) CDRs and a human constant region. It should be understood that the framework regions can be derived from one source or more than one source and that the CDRs can be derived from one source or more than one source.

The antibody containing the engineered heavy chain constant region is selected based on its ability to bind to a cell surface molecule or a soluble molecule that binds to a cell surface molecule. Thus, for example, the antibody can be selected based on its ability to bind cell surface molecules such as cytokine receptors (e.g., IL-2R, TNF-αR, IL-15R, etc.); adhesion molecules (e.g., E-selectin, P-selectin, L-selectin, VCAM, ICAM, etc.); cell differentiation or activation antigens (e.g., CD3, CD4, CD8, CD20, CD25, CD40, etc.), and others. Alternatively, the antibody can be selected based on its ability to bind a soluble molecule that binds to cell surface molecules. Such soluble molecules include, but are not limited to, cytokines and chemokines (e.g., interleukin-1 (IL-1), IL-2, IL-3, IL-5, IL-6, etc.); growth factors (e.g., EGF, PGDF, GM-CSF, HGF, IGF, etc.); molecules inducing cell differentiation (e.g., EPO, TPO, SCF, PTN, etc.), and others.

The term “antibody” as used herein includes whole antibodies and antibody fragments that include at least two of CH1, hinge region, CH2 or CH3. Whole monoclonal antibodies are preferred.

In general, the construction of the antibodies disclosed herein is achieved by using recognized manipulations utilized in genetic engineering technology. For example, techniques for isolating DNA, making and selecting vectors for expressing the DNA, purifying and analyzing nucleic acids, specific methods for making recombinant vector DNA, cleaving DNA with restriction enzymes, ligating DNA, introducing DNA including vector DNA into host cells by stable or transient means, culturing the host cells in selective or non-selective media to select and maintain cells that express DNA, are generally known in the field.

The monoclonal antibodies disclosed herein may be derived using the hybridoma method (Kohler et al., Nature, 256:495, 1975), or other recombinant DNA methods well known in the art. In the hybridoma method, a mouse or other appropriate host animal is immunized with a protein which elicits the production of antibodies by the lymphocytes. Alternatively, lymphocytes may be immunized in vitro. The lymphocytes produced in response to the antigen are then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells are then seeded and grown in a suitable culture medium that preferably contains one or more substances that inhibit the growth or survival of the unfused, parental myeloma cells. Preferred myeloma cells are those that fuse efficiently, support stable production of antibody by the selected antibody-producing cells, and are not sensitive to a medium such as HAT medium (Sigma Chemical Company, St. Louis, Mo., Catalog No. H-0262). Among these, preferred myeloma cell lines are murine myeloma lines, such as those derived from MOPC-21 and MPC-11 mouse tumors available from the Salk Institute Cell Distribution Center, San Diego, Calif. USA, and SP-20, NS0 or X63-Ag8-653 cells available from the American Type Culture Collection, Rockville, Md. USA.

The hybridoma cells are grown in a selective culture medium (e.g., HAT) and surviving cells expanded and assayed for production of monoclonal antibodies directed against the antigen. The binding specificity of monoclonal antibodies produced by hybridoma cells may be determined by assays such as immunoprecipitation, radioimmunoassay (RIA), flow cytometry, cell activation assays or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, or mammalian cells that do not otherwise produce immunoglobulin proteins, to obtain the synthesis of monoclonal antibodies in the recombinant host cells. Antibodies or antibody fragments can also be isolated from antibody phage libraries generated using the techniques described in McCafferty et al., Nature, 348:552-554 (1990). Other publications have described the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The antibodies described herein are then modified by combining the coding sequence for the present engineered IgG2/IgG4-derived human heavy-chain constant domains with the coding sequence for a heavy chain variable domain. Where the present recombinant antibody is based on a particular murine antibody, for example, an engineered heavy chain constant region in accordance with this disclosure can be substituted in place of the homologous murine sequences. Alternatively, a functional antibody fragment can be identified (e.g., through the panning of a human phage library, an scFv library or a Fab library) onto which the present engineered IgG2/IgG4-derived human heavy-chain constant domains can be engineered.

In another aspect, this disclosure provides recombinant expression vectors which include the synthetic, genomic or cDNA-derived nucleic acid fragments necessary to produce the engineered heavy chain constant region. The nucleotide sequence coding for any of the engineered heavy chain constant regions or antibodies containing the engineered heavy chain constant regions in accordance with this disclosure can be inserted into an appropriate vector which contains the necessary elements for the transcription and translation of the inserted protein-coding sequence. Any suitable host cell vector may be used for expression of the DNA sequences coding for the chimeric or CDR-grafted heavy and light chains. Bacterial (e.g. E.coli) and other microbial systems may be used. Eukaryotic (e.g. mammalian) host cell expression systems may also be used to obtain antibodies of the present invention. Suitable mammalian host cell include COS cells and CHO cells (Bebbington C R (1991) Methods 2 136-145); and myeloma or hybridoma cell lines (for example NSO cells (Bebbington, et al., Bio Technology, 10: 169-175 (1992)).

The antibodies containing the engineered heavy chain constant region can also be used as separately administered compositions given in conjunction with therapeutic agents. For diagnostic purposes, the antibodies may either be labeled or unlabeled. Unlabeled antibodies can be used in combination with other labeled antibodies (second antibodies) that are reactive with the engineered antibody, such as antibodies specific for human immunoglobulin constant regions. Alternatively, the antibodies can be directly labeled. A wide variety of labels may be employed, such as radionuclides, fluors, enzymes, enzyme substrates, enzyme co-factors, enzyme inhibitors, ligands (particularly haptens), etc. Numerous types of immunoassays are available and are well known to those skilled in the art.

The present engineered antibodies can be administered to a patient in a composition comprising a pharmaceutical carrier. A pharmaceutical carrier can be any compatible, non-toxic substance suitable for delivery of the antibodies to the patient. Sterile water, alcohol, fats, waxes, and inert solids may be included in the carrier. Pharmaceutically accepted adjuvants (buffering agents, dispersing agent) may also be incorporated into the pharmaceutical composition.

The antibody compositions may be administered to a patient in a variety of ways. Preferably, the pharmaceutical compositions may be administered parenterally, e.g., subcutaneously, intramuscularly or intravenously. Thus, compositions for parental administration may include a solution of the antibody, antibody fragment or a cocktail thereof dissolved in an acceptable carrier, preferably an aqueous carrier. A variety of aqueous carriers can be used, e.g., water, buffered water, 0.4% saline, 0.3% glycine, etc. These solutions are sterile and generally free of particulate matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. The concentration of antibody or antibody fragment in these formulations can vary widely, e.g., from less than about 0.5%, usually at or at least about 1% to as much as 15 or 20% by weight and will be selected primarily based on fluid volumes, viscosities, etc., in accordance with the particular mode of administration selected.

Actual methods for preparing parenterally administrable compositions and adjustments necessary for administration to subjects will be known or apparent to those skilled in the art and are described in more detail in, for example, Remington's Pharmaceutical Science, 17^{th} Ed., Mack Publishing Company, Easton, Pa. (1985), which is incorporated herein by reference.

In another embodiment the present disclosure provides fusion proteins that include a non-Fc component fused to an Fc region that is engineered to include an IgG2-derived portion and an IgG4-derived portion. The Fc region can be an engineered constant region in accordance with any of the various embodiments described above with respect to antibodies. The Fc region can include all or any portion of a CH1, hinge, CH2 and CH3 domain, provided that the Fc region includes at least one IgG2-derived portion and at least one IgG4-derived portion. Preferably, the IgG2-derived portion includes at least the heavy chain constant region 1 and hinge region and the IgG4-derived portion includes most of the heavy chain constant region 2 and the entire heavy chain constant region 3. A linker may optionally be provided between the non-Fc component and the Fc region. The linker, when present, can be from 3 to 25 amino acids long. In particularly useful embodiments, the linker assists in maintaining proper folding and therefore function of the non-Fc component.

Preferably the fusion proteins in accordance with this disclosure maintain the function of the non-Fc portion of the protein and/or have increased half-life compared to the non-Fc portion alone. In addition, the fusion proteins in accordance with this disclosure preferably lack unwanted antibody Fc-mediated cell activating and inflammatory properties including events resulting from Fc-receptor antibody engagement and complement activation. Also, the fusion proteins in accordance with certain embodiments of this disclosure which include the hinge region have the ability to form disulfide bonds with other fusion protein molecules, thereby resulting in dimer formation which may increase the avidity of the non-Fc portion for the molecule to which it binds.

Fusion proteins in accordance with this disclosure may be readily secreted in stable form by mammalian cells transfected with DNA that codes for the molecule. In addition, they are amenable to rapid, efficient purification to homogeneity, for example, using protein A. Because these molecules therefore are obtainable in a commercially useful amount and form, they are advantageous substitutes for monoclonal antibodies in contexts such as flow cytometry, immunohistochemistry, immunoprecipitation, cell-based assays and enzyme-linked immunoadsorbant assays (ELISAs).

Any peptide or protein that exhibits a useful property is suitable for use as the non-Fc component to be combined with the present engineered antibody constant region to prepare the fusion protein. Peptide or protein activities and uses include, but are not limited to, serving as a protein agonist or antagonist, binding a receptor, binding a membrane bound surface molecule, binding a soluble protein, binding a ligand, binding an enzyme or structural protein, activating or inhibiting a receptor, targeted drug delivery, or any enzymatic activity. Those peptides or proteins whose utility can be increased from the enhanced stability and half-life conferred upon them when presented in combination with an Fc domain are usually selected. It should be understood that “biological activity” as used herein includes any activity associated with a molecule having activity in a biological system, including, but not limited to, the stimulatory or inhibitory activity triggered by protein-protein interactions as well as the kinetics surrounding such interactions including the stability of a protein-protein complex. Thus, non-limiting examples of molecules suitable for use as the non-Fc component include cytokines, hormones, enzymes, ligands, growth factors, receptors and antibody fragments.

Suitable non-Fc components suitable for use in preparing the present fusion proteins include: peptides that bind to receptors which are activated by ligand-induced homo-dimerization including active fragments displaying G-CSF activity, GHR activity and prolactin activity as described in Whitty and Borysenko, Chem Biol., April (1999) 6(4):R107-18; other examples of suitable peptides include a nerve growth factor mimetic from the CD loop as described in Zaccaro et al., Med. Chem. (2000) 43(19); 3530-40; an IL-2 mimetic as described in Eckenberg, et al., J. Immunol. (2000) 165(8):4312-8; glucogon-like peptide-1 as described in Evans et al., Drugs R.D. (1999) 2(2): 75-94; tetrapeptide I (D-lysine-L-asparaginyl-L-prolyl-L-tyrosine) which stimulates mitogen activated B cell proliferation as described in Gagnon et al., Vaccine (2000) 18(18):1886-92; the binding domain of human cytotoxic T-lymphocyte-associated antigen 4. Peptides which exhibit receptor antagonistic activity are also contemplated. For example, N-terminal peptide of vMIP-II as an antagonist of CXCR4 for HIV therapy as described in Luo et al., Biochemistry (2000) 39(44):13545-50; antagonist peptide ligand (AFLARAA) of the thrombin receptor for antithrombotic therapy as described in Pakala et al., Thromb. Res. (2000) 100(1): 89-96; peptide CGRP receptor antagonist CGRP (8-37) for attenuating tolerance to narcotics as described in Powell et al., Br. J. Pharmacol. (2000) 131(5): 875-84; parathyroid hormone (PTH)-1 receptor antagonist known as tuberoinfundibular peptide (7-39) as described in Hoare et al., J. Pharmacol. Exp. Ther. (2000) 295(2):761-70; opioid growth factor as described in Zagon et al., Int. J. Oncol. (2000) 17(5): 1053-61; high affinity type I interleukin 1 receptor antagonists as disclosed in Yanofsky, et al., Proc. Natl. Acad. Sci. USA, Vol. 93, pp. 7381-7386, July 1996 and Vigers, et al., J. Biol. Chem., Vol 275, No 47, pages 36927-36933, 2000; and acid fibroblast growth factor binding peptide as described in Fan et al., IUBMB Life (2000) 49 (6) 545-48. Further examples of biologically active peptides which can be fused with a Fc region in accordance with this disclosure include proteins secreted by the heart as part of the body's response to congestive heart failure, such as, for example, human brain natriuretic peptide (hBNP) as described in Mukoyama, et al., J. Clin. Invest. 87(4): 1402-12 (1991) and Clemens, et al., J. Pharmacol. Exp. Ther. 287(1): 67-71(1998). Additional examples of biologically active peptides which can be used in accordance with this disclosure include proteins which have the potential to preserve or improve beta-cell function (e.g., by inducing glucose-dependent insulinotropic effect), such as, for example, exendin-4, GLP-1 (7-36), GPL-2 (1-34), glucagons or PACAP-38 (see, Raufman, et al., J. Biol. Chem. 267(30): 21432-7 (1992). It should also be understood that antibody fragments can also be employed as the non-Fc component of the fusion protein. Thus, for example, the non-Fc component can be an sc-Fv, F(ab) or F(ab)¹₂. As another example, the non-Fc component can be an antibody variable region into which a mimetic peptide has been inserted into or in place of one or more CDR regions as described in WO 02/46238A2, the disclosure of which is incorporated herein in its entirety by this reference.

Thus, for example, the non-Fc component used to make the fusion protein can be a growth factor. Examples of growth factors include platelet-derived growth factor (PDGF), keratinocyte growth factor (KGF), epidermal growth factor (EGF), vascular endothelial growth factor (VEGF), insulin, nerve growth factor (NGF), insulin-like growth factor (IGF), transforming growth factor (TGF), hepatic growth factor (HGF), fibroblast growth factor (FGF), the product of the Wnt-2 proto-oncogne (wnt-2). Aaronson, supra; Norman et al., HORMONES, pp. 719-748 (Academic Press 1987). Also, see generally, Heath (ed.), GROWTH FACTORS, IRL Press (1990).

### I. Production of Fusion Proteins

A. Construction of Fusion Protein Expression Vectors

Any technique within the purview of those skilled in the art can be used to produce fusion proteins which comprise an IgG2-derived portion and an IgG4-derived portion and a non-Fc portion. Suitable techniques include, but are not limited to those methods disclosed in U.S. Pat. Nos. 5,670,625; 5,726,044; and 6,403,769. In one such technique, where the fusion protein is secreted in stable form by mammalian cells, DNA sequences coding for the fusion protein are subcloned into an expression vector which is used to transfect mammalian cells. General techniques for producing fusion proteins comprising antibody sequences are described in Coligan et al. (eds.), CURRENT PROTOCOLS IN IMMUNOLOGY, at pp. 10.19.1-10.19.11 (Wiley Interscience 1992), the contents of which are hereby incorporated by reference. See also METHODS: A COMPANION TO METHODS IN ENZYMOLOGY, Volume 2 (No. 2), Academic Press (1991), and ANTIBODY ENGINEERING: A PRACTICAL GUIDE, W.H. Freeman and Company (1992), in which commentary relevant to production of fusion proteins is dispersed throughout the respective texts. The present methods are not limited to any particular method of expression. Expression thus can be achieved using eukaryotic (e.g., mammalian, insect) or prokaryotic (e.g., bacteria) cells, and the fusion proteins can be secreted by the cells or recovered from periplasm or inclusion bodies within the cells.

Thus, one of the steps in the construction of fusion proteins is to subclone portions of the fusion proteins into cloning vectors. In this context, a “cloning vector” is a DNA molecule, such as a plasmid, cosmid or bacteriophage, that can replicate autonomously in a host prokaryotic cell. Cloning vectors typically contain one or a small number of restriction endonuclease recognition sites at which foreign DNA sequences can be inserted in a determinable fashion without loss of an essential biological function of the vector, as well as a marker gene-that is suitable for use in the identification and selection of cells transformed with the cloning vector. Marker genes typically include genes that provide tetracycline resistance or ampicillin resistance. Suitable cloning vectors are described by Sambrook et al. (eds.), MOLECULAR CLONING: A LABORATORY MANUAL, Second Edition (Cold Spring Harbor Press 1989) (hereafter “Sambrook”); by Ausubel et al. (eds.), CURRENT PROTOCOLS IN MOLECULAR BIOLOGY (Wiley Interscience 1987) (hereafter “Ausubel”); and by Brown (ed.), MOLECULAR BIOLOGY LABFAX (Academic Press 1991). Suitable cloning vectors are commercially available.

The DNA sequence encoding the Fc region of the fusion protein can be obtained using any technique within the purview of those skilled in the art. DNA sequences encoding the non-Fc portion of the fusion protein can also be synthesized using techniques within the purview of those skilled in the art, such as PCR with RNA isolated from cells that produce the non-antibody protein. The DNA can include introns or can be engineered to remove some or all introns.

DNA sequences encoding the non-Fc component of the fusion protein are subcloned in frame with the N-terminus of the Fc region portion of the fusion protein Subcloning is performed in accordance with techniques within the purview of those skilled in the art, such as the use of restriction enzyme digestion to provide appropriate termini, the use of alkaline phosphatase treatment to avoid undesirable joining of DNA molecules, and ligation with appropriate ligases. Techniques for such manipulation are described by Sambrook and Ausubel, and are well-known in the art. Techniques for amplification of cloned DNA in bacterial hosts and isolation of cloned DNA from bacterial hosts also are well-known.

It should, of course be understood that the Fc region can be cloned onto the amino terminus of the non-Fc component, if desired.

The cloned fusion protein is cleaved from the cloning vector and inserted into an expression vector. Suitable expression vectors typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence.

A fusion protein in accordance with this disclosure can be expressed in eukaryotic cells, such as mammalian, insect and yeast cells. Mammalian cells are especially preferred eukaryotic hosts because mammalian cells provide suitable post-translational modifications such as glycosylation. Examples of mammalian host cells include Chinese hamster ovary cells (CHO-K1; ATCC CCL61), rat pituitary cells (GH₁; ATCC CCL82), HeLa S3 cells (ATCC CCL2.2), rat hepatoma cells (H-4-II-E; ATCC CRL1548) SV40-transformed monkey kidney cells (COS-1; ATCC CRL 1650) and murine embryonic cells (NIH-3T3; ATCC CRL 1658).

For a mammalian host, the transcriptional and translational regulatory signals may be derived from viral sources, such as adenovirus, cytomegalovirus, bovine papilloma virus, simian virus, or the like, in which the regulatory signals are associated with a particular gene which has a high level of expression. Suitable transcriptional and translational regulatory sequences also can be obtained from mammalian genes, such as actin, collagen, myosin, and metallothionein genes.

Transcriptional regulatory sequences include a promoter region sufficient to direct the initiation of RNA synthesis. Suitable eukaryotic promoters include the promoter of the mouse metallothionein I gene (Hamer et al., J. Molec. Appl. Genet. 1: 273 (1982)]; the TK promoter of herpes virus (McKnight, Cell 31: 355 (1982)); the SV40 early promoter (Benoist et al., Nature 290: 304 (1981)); the Rous sarcoma virus promoter (Gorman et al., Proc. Nat'l Acad. Sci. USA 79: 6777 (1982)); and the cytomegalovirus promoter (Foecking et al., Gene 45: 101 (1980)).

Alternatively, a prokaryotic promoter, such as the bacteriophage T3 RNA polymerase promoter, can be used to control fusion gene expression if the prokaryotic promoter is regulated by a eukaryotic promoter. Zhou et al., Mol. Cell. Biol. 10: 4529 (1990); Kaufman et al., Nucl. Acids Res. 19: 4485 (1991).

An expression vector can be introduced into host cells using a variety of techniques including calcium phosphate transfection, liposome-mediated transfection, electroporation, and the like. Preferably, transfected cells are selected and propagated wherein the expression vector is stably integrated in the host cell genome to produce stable transformants. Techniques for introducing vectors into eukaryotic cells and techniques for selecting stable transformants using a dominant selectable marker are described by Sambrook, by Ausubel, by Bebbington, “Expression of Antibody Genes in Nonlymphoid Mammalian Cells,” in 2 METHODS: A COMPANION TO METHODS IN ENZYMOLOGY 136 (1991), and by Murray (ed.), GENE TRANSFER AND EXPRESSION PROTOCOLS (Humana Press 1991).

Stable transformants that produce a fusion protein can be identified using a variety of methods. For example, stable transformants can be screened using an antibody that binds either to the non-antibody portion of the fusion protein or to the antibody portion of the fusion protein. The use of immunoprecipitation to identify cells is a technique well known to those skilled in the art.

After fusion protein-producing cells have been identified, the cells are cultured and fusion proteins are isolated from culture supernatants. Suitable isolation techniques include affinity chromatography with Protein-A Sepharose, size-exclusion chromatography and ion exchange chromatography. Protein A is a particularly useful way to isolate fusion proteins from supernatants.

Routine assays can be performed to determine whether the non-Fc portion of the fusion protein retains it functionality.

Fusion proteins can be detectably labeled with any appropriate marker moiety, for example, a radioisotope, an enzyme, a fluorescent label, a chemiluminescent label, a bioluminescent labels or a paramagnetic label. Methods of making and detecting such detectably-labeled fusion proteins are well-known to those of ordinary skill in the art.

In vitro and in situ detection methods may be used to assist in the diagnosis or staging of a pathological condition. The present disclosure also contemplates the use of fusion proteins for in vivo diagnosis.

The fusion proteins in accordance with this disclosure can be formulated into pharmaceutically acceptable compositions and administered in the manner described above for the antibody embodiments.

The following examples are intended to illustrate but not limit the invention. While they are typical of those that might be used, other procedures known to those skilled in the art may alternatively be used.

### EXAMPLE 1

Anti-CD3 Antibody with an Engineered Heavy Chain Constant Region

The variable heavy chain of OKT3 was joined through standard recombinant DNA methodology to a genomic DNA cassette containing the first heavy chain constant region (CH1), the hinge linker region, and the first few amino acids from the second heavy chain constant region (CH2) from human IgG2. Next, a cassette containing the balance of the second heavy chain constant region (CH2) and third heavy chain constant region (CH3) from human IgG4 was added. The IgG2 hinge region and following amino acids were chosen to minimize antibody binding to Fc-gamma receptors and the IgG4 regions were chosen to prevent antibody-mediated complement activation.

Preparation of Engineered Heavy Chain Constant Region

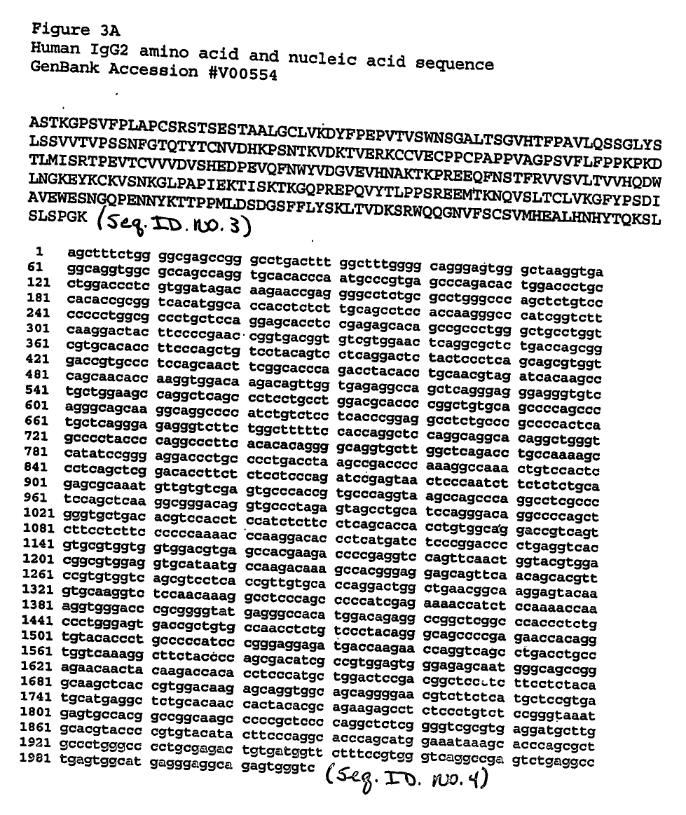

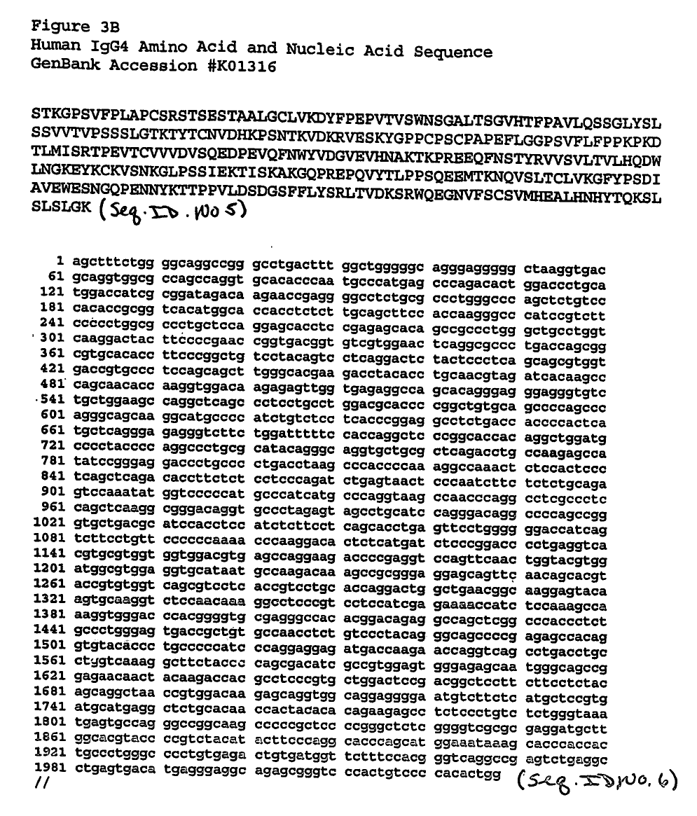

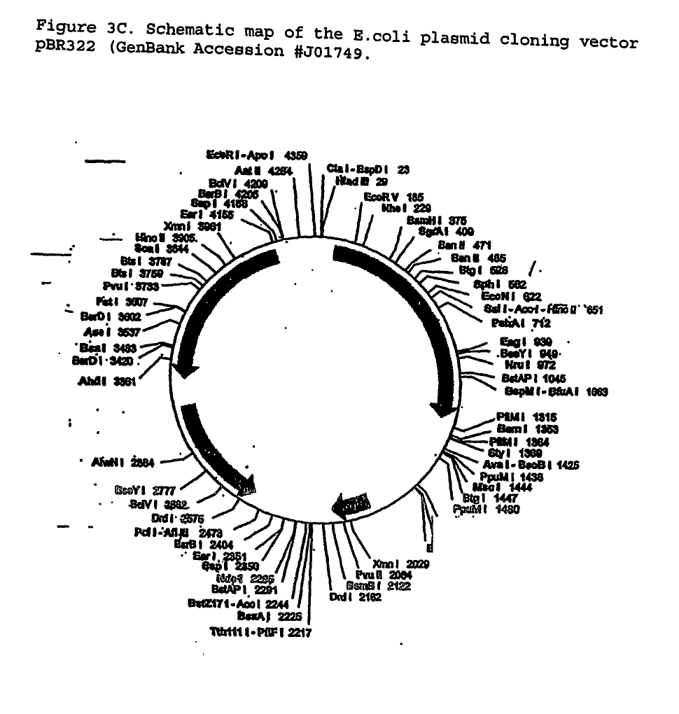

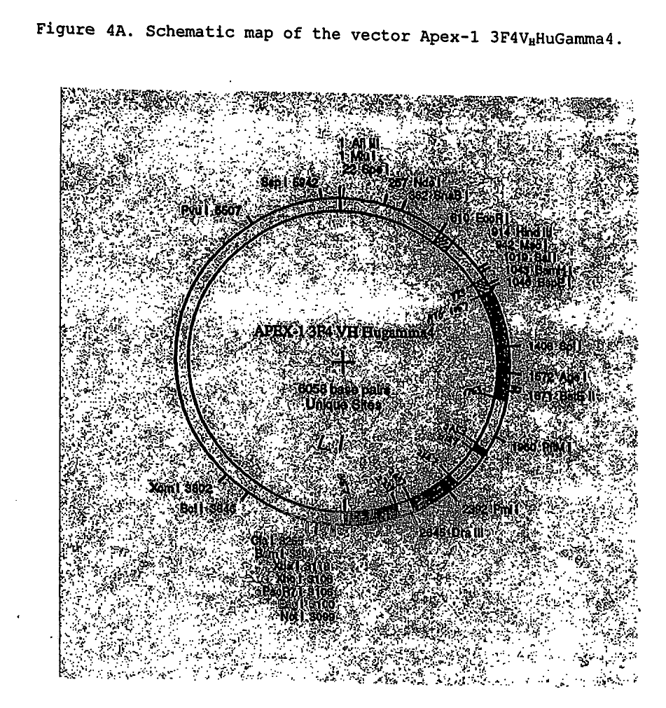

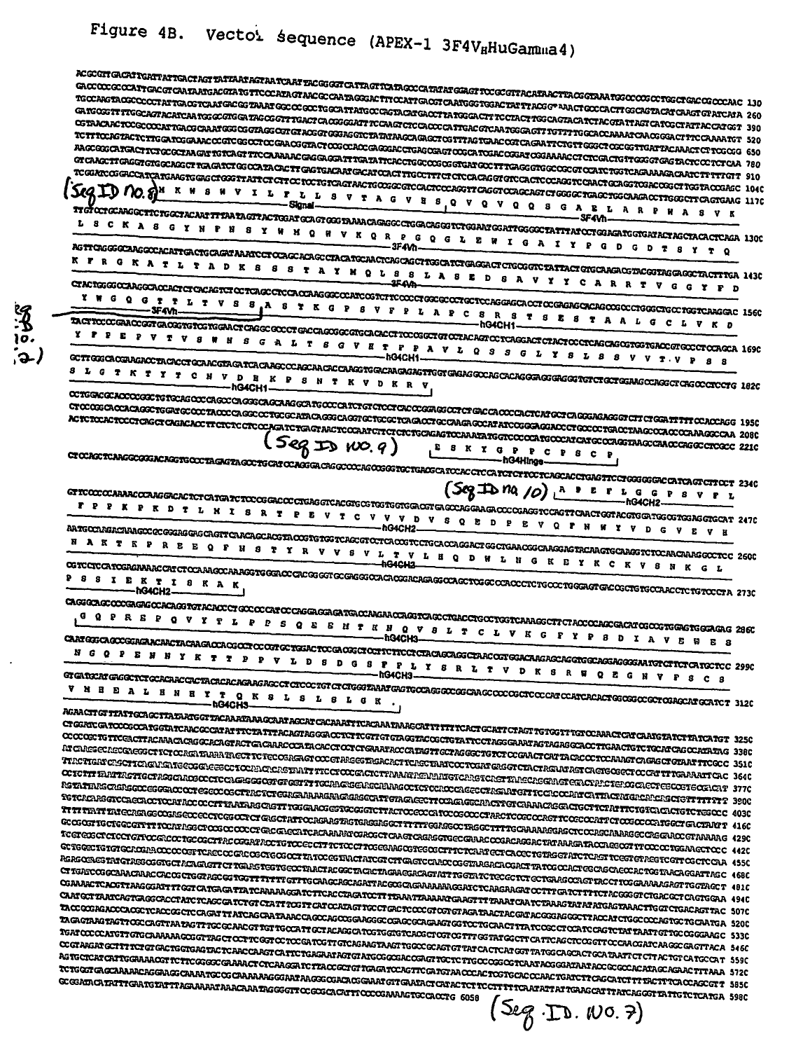

Genomic DNA encoding either the human IgG2 heavy chain constant region (GenBank accession # V00554; see FIG. 3A) or the human IgG4 heavy chain constant region (GenBank accession # K01316; see FIG. 3B) were provided, as inserts in the bacterial carrier plasmid pBR322 (see FIG. 3C), by Dr. Ed Max of the FDA. Restriction enzyme analysis and complete DNA sequencing confirmed that the correct sequences of human IgG4 and IgG2 constant regions were obtained. The IgG4-derived inserts were released from the plasmid by performing restriction digests with HindIII and XhoI. The inserts were purified, excised, and subjected to further restriction analysis to confirm the published sequence of the human IgG4 genomic DNA. The genomic IgG4 insert (HindIII/SmaI restriction fragment; the SmaI site is in the 3′ untranslated region approximately 30 bp 3′ of the translation stop site) was then subcloned by ligation into the expression cassette APEX-1 (see FIGS. 4A and 4B, APEX-1 3F4V_{H}HuGamma4). DNA sequence analysis was performed to confirm the correct sequence of the human IgG4 desired regions.

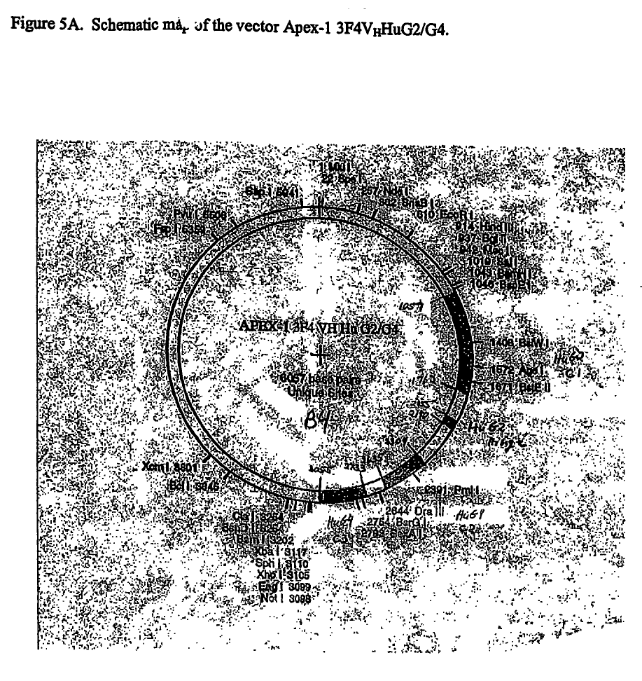

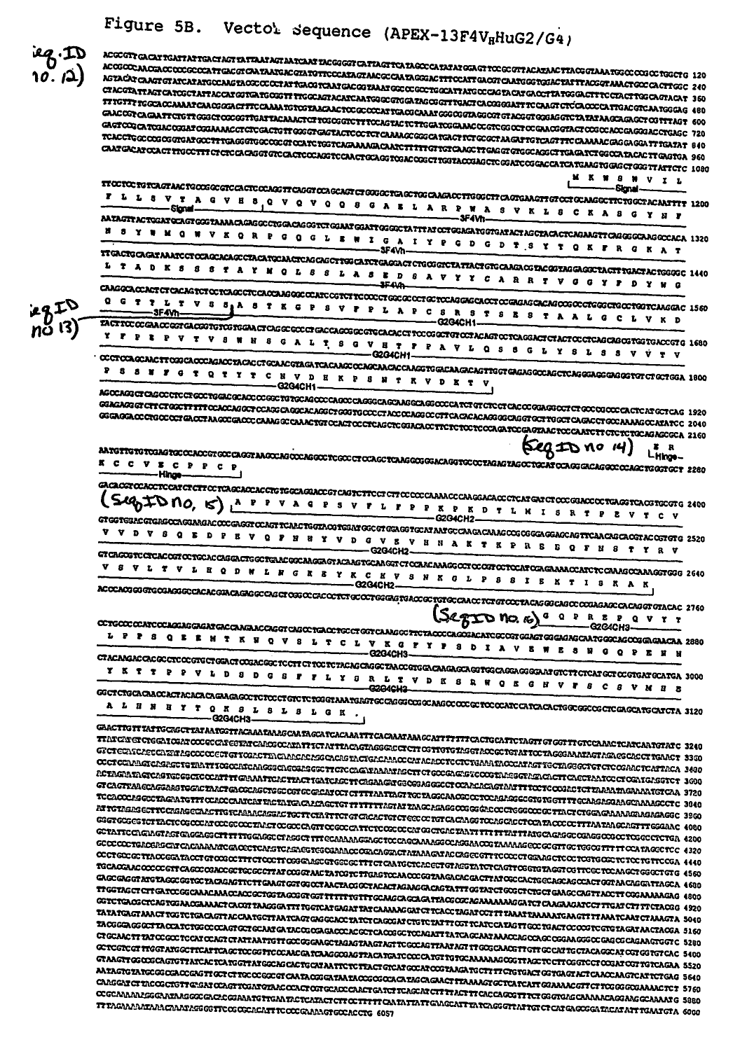

The pBR322 plasmid containing genomic DNA encoding human IgG2 was used as the source of IgG2 CH1, hinge region and the first part of CH2, which were excised with PmII and Bst EII and subcloned into APEX-1 3F4V_{H}Gamma4 to replace the corresponding IgG4 derived sequences (see FIG. 5A). The sequence of the resulting chimeric IgG2/IgG4 human constant region is shown in FIG. 5B (APEX-1 3F4V_{H}HuG2/G4).

Preparation of a Chimeric Antibody Based Upon Murine OKT3 Variable Regions and the Human G2/G4 Heavy Chain Constant Region

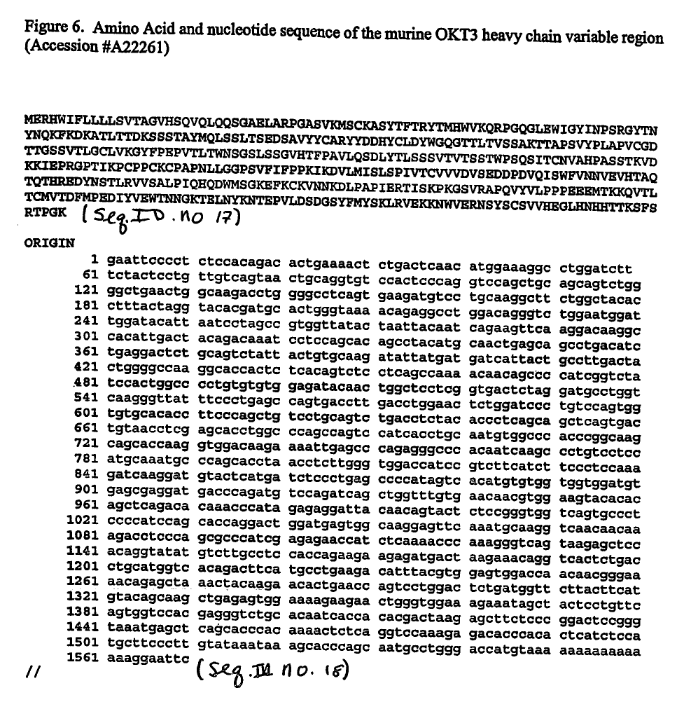

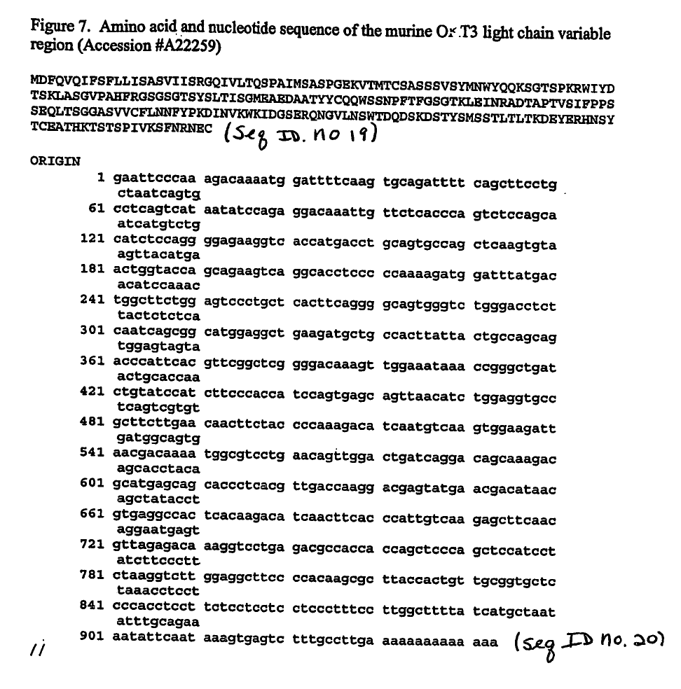

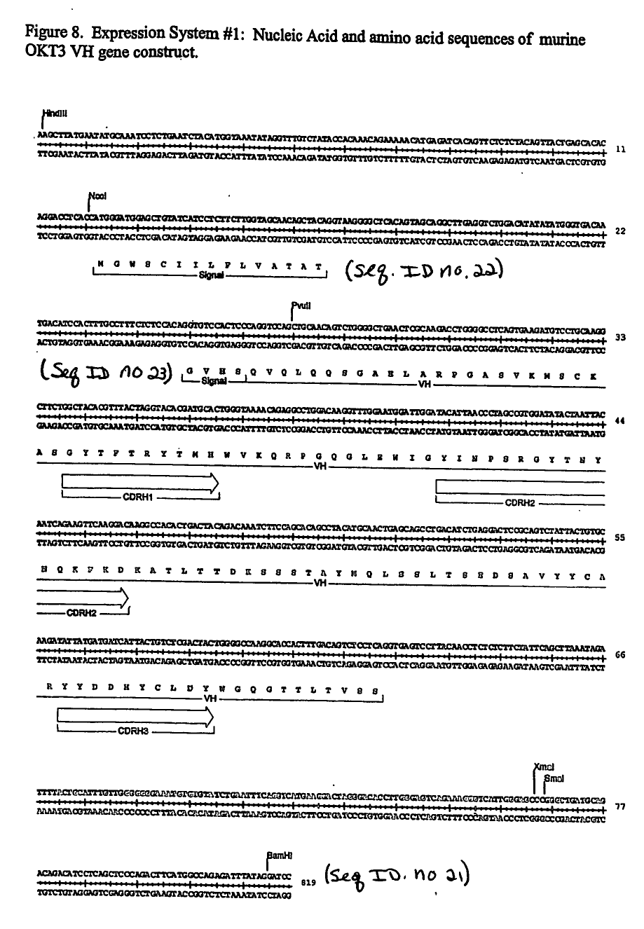

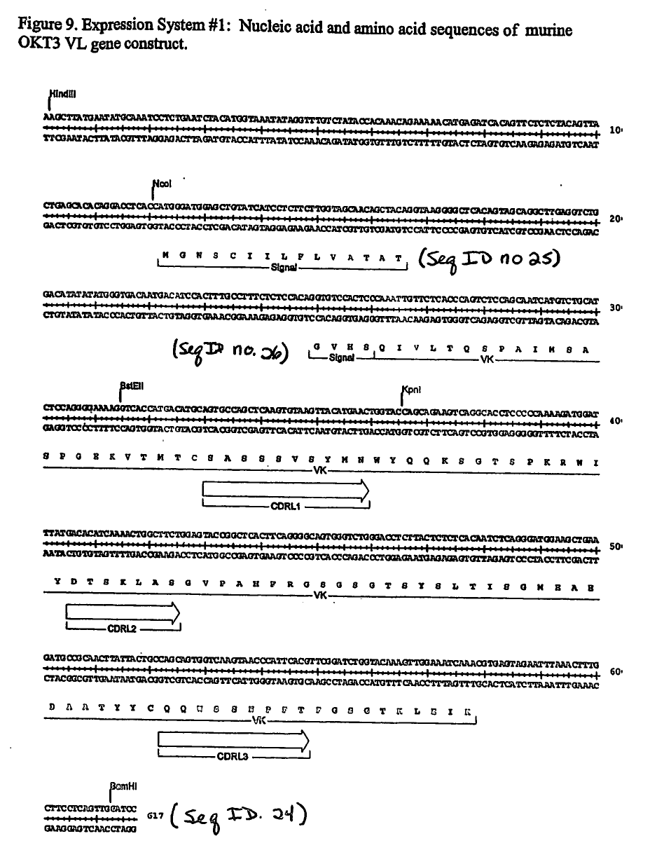

The murine mAb OKT3 heavy and light chain variable sequences have been previously determined and deposited in the GenBank database (Accession numbers A22261 (FIG. 6) and A22259 (FIG. 7), respectively). A chimeric antibody was generated with the OKT3 variable regions and the HuG2/G4 constant region using two different expression systems. The heavy and light chain variable regions were constructed by gene synthesis using overlapping 40 mer oligonucleotides and a ligase chain reaction for insertion into PUC 19 cloning vectors. For Expression System #1, sequences including the murine immunoglobulin promoter and a murine leader sequence with the leader intron, were added at the 5′ ends, and sequences including the splice donor site were added at the 3′ ends by PCR to form expression cassettes for, the heavy and light chain (kappa) variable regions as HindIII to BamHI fragments. The complete DNA and amino acid sequences of the constructed murine OKT3 heavy chain variable region and the murine OKT3 light chain variable region used in Expression System #1 are shown in FIGS. 8 and 9, respectively.

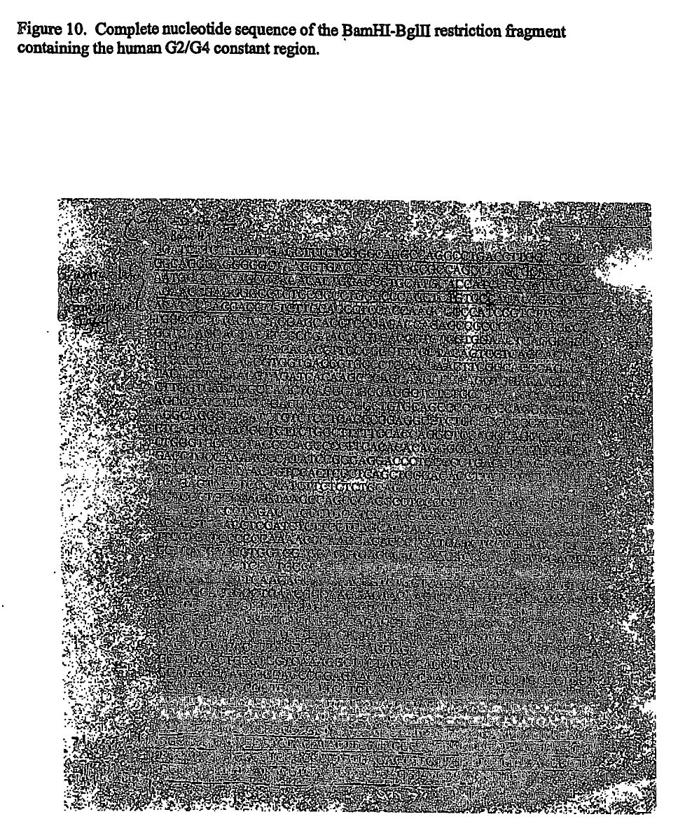

The previously described engineered heavy chain constant region was next modified for insertion into a separate PUC1 9 cloning vector as follows: 5′ untranslated intron sequence from native human IgG4 with a 5′ BamHI site was added at the 5′ end, and 3′ untranslated sequence from natural human IgG4 with 3′ EcoRI and BglII sites was added at the 3′ end. The HuG2/G4 constant region was excised as a BamHI to BglII fragment from Puc 19, inserted into the unique BamHI site of the heavy chain expression vector pSVgpt.HuG2G4 and the correct orientation selected. The complete nucleic acid sequence of the BamHI to BglII HuG2/G4 fragment is shown in FIG. 10.

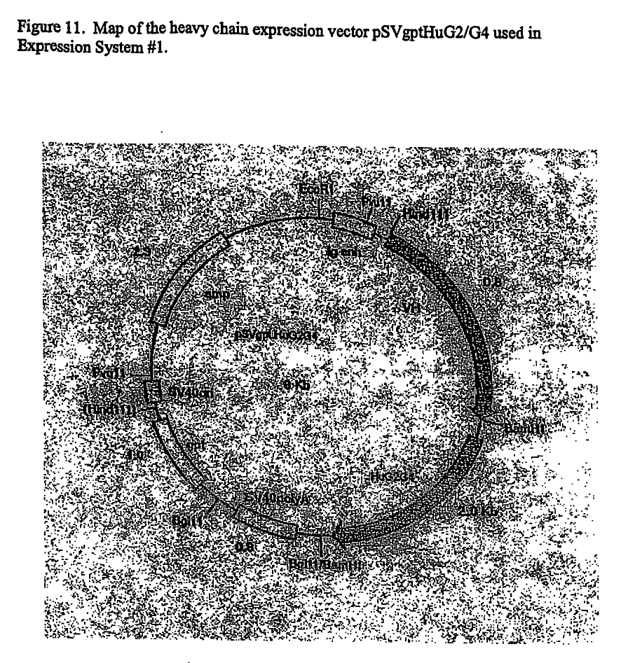

Similarly, the constructed murine OKT3 heavy chain variable region was excised from PUC 19 as a HindIII to BamHI fragment and transferred to the pSVgpt.HuG2G4 expression vector containing the HuG2/G4 insert. The DNA sequence was confirmed to be correct. A schematic map of the heavy chain expression vector pSVgpt.HuG2G4 is shown in FIG. 11, and indicates the position of the HuG2/G4 constant region relative to the constructed OKT3 variable heavy region contained within the vector.

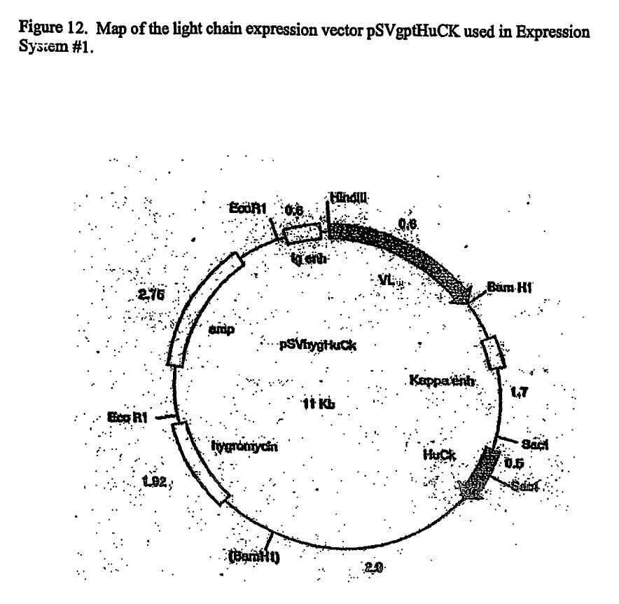

The constructed murine OKT3 light chain variable region was also excised from PUC 19 as a HindIII to BamHI fragment and transferred to the expression vector pSVhygHuCK containing the human kappa constant region (HuCk) as shown in FIG. 12.

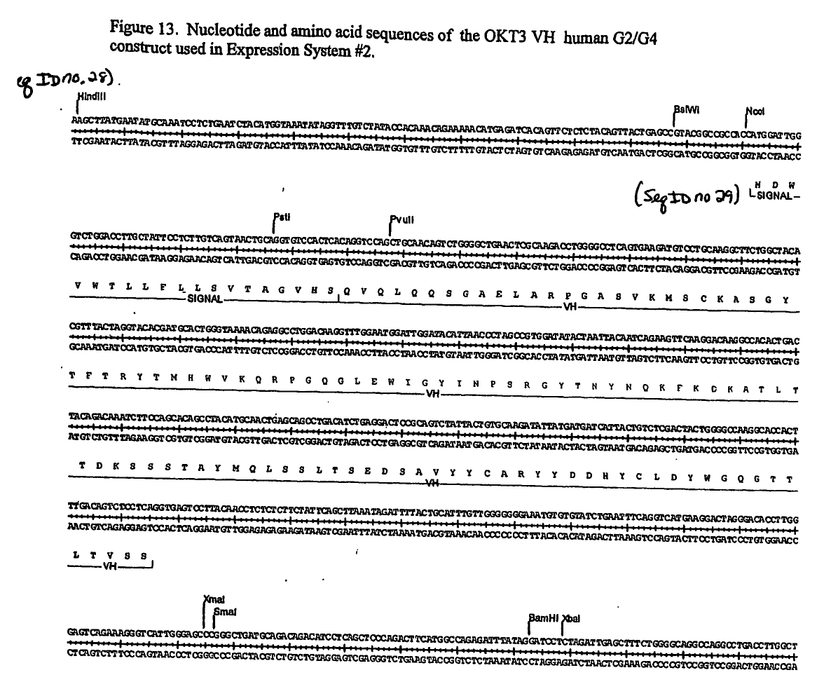

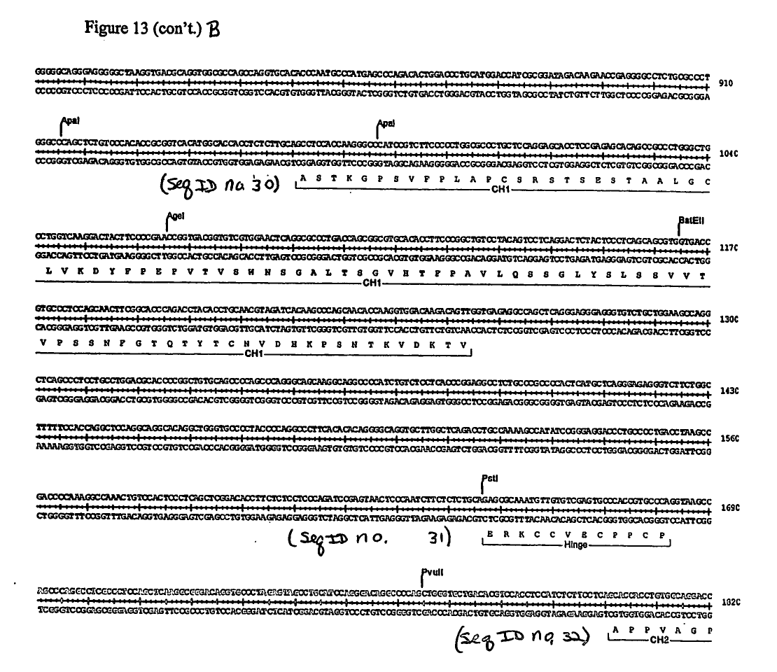

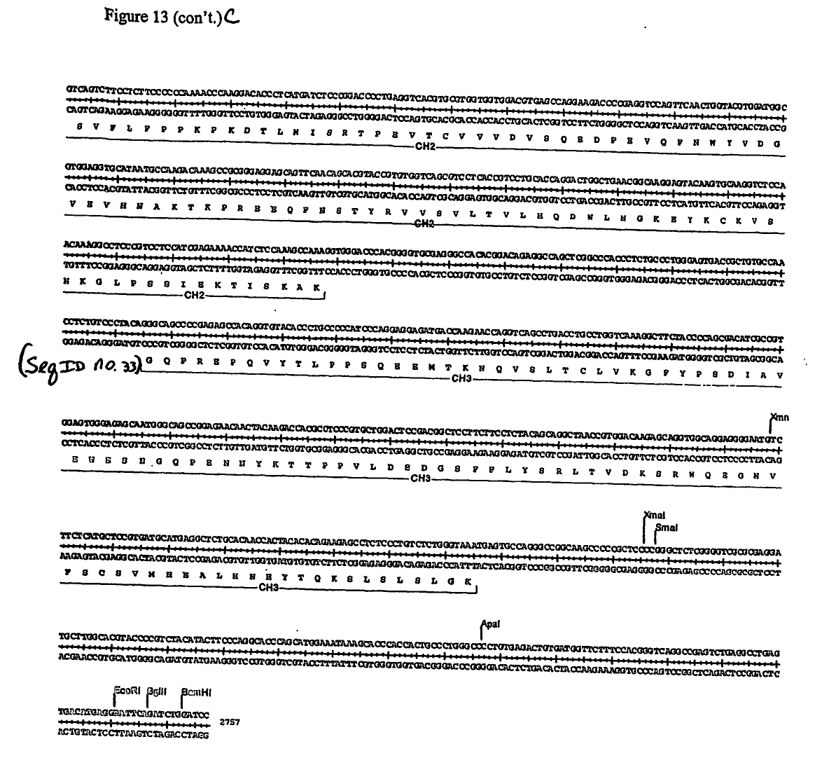

A second expression system containing a modified version of the murine OKT3 variable heavy chain region joined to the human G2/G4 constant region was also generated. This version (Expression System #2) included the original OKT3 signal sequence and did not contain the immunoglobulin promoter and intron sequences described in the previous construct. The chimeric antibody was constructed by gene synthesis and ligated into the PUC 19 cloning vector containing the previously described G2/G4 constant region. The sequences of the OKT3 VH and human G2/G4 inserts (Expression System #2) are shown in FIG. 13.

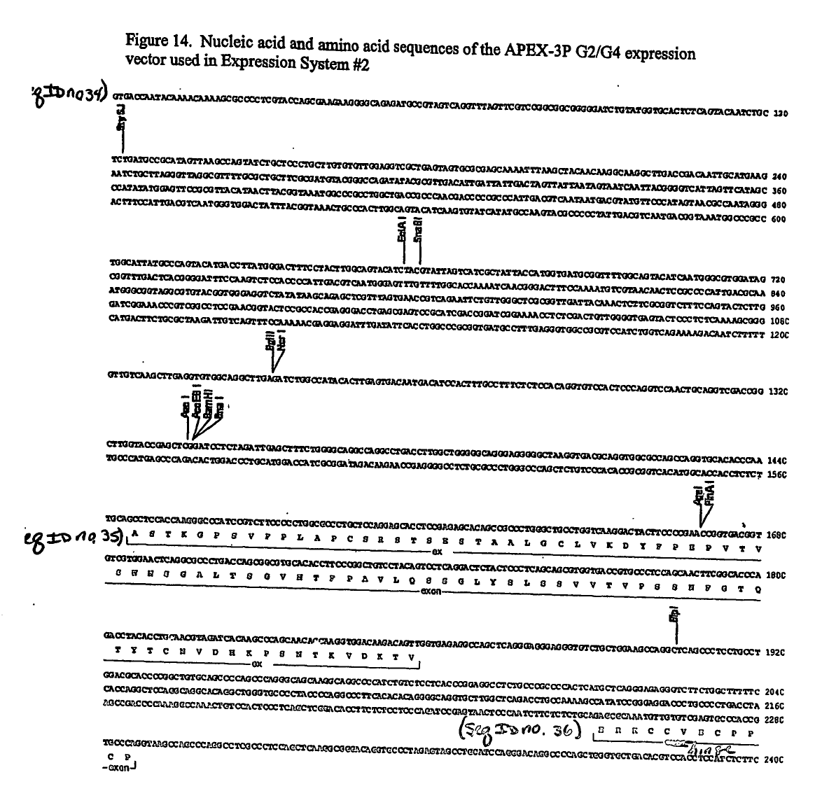

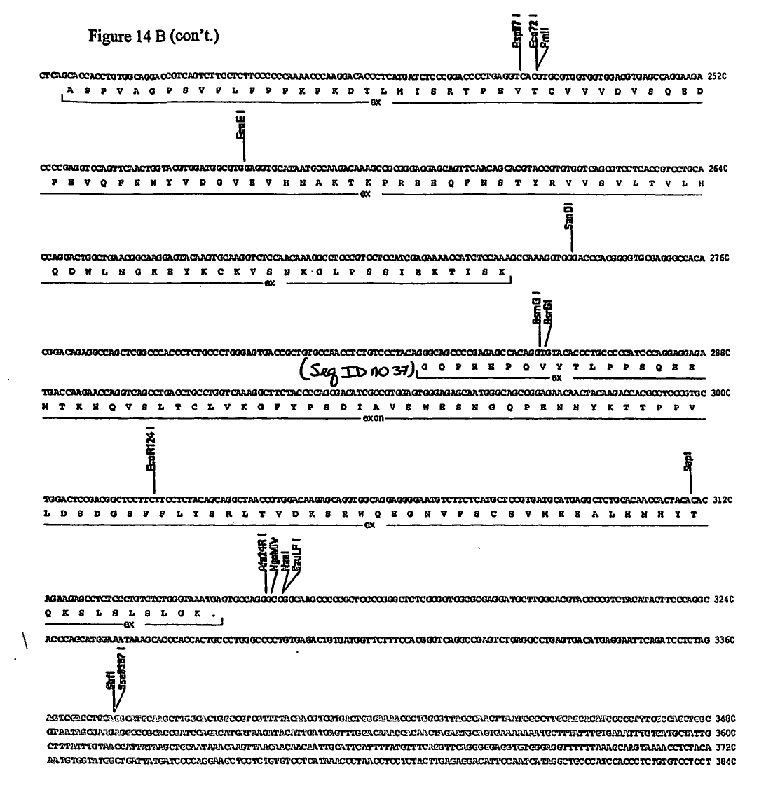

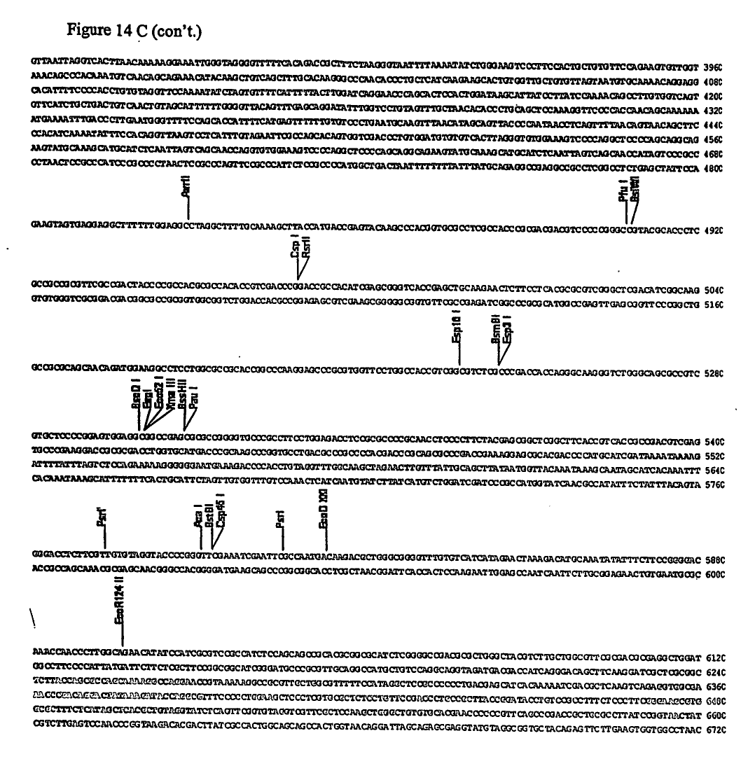

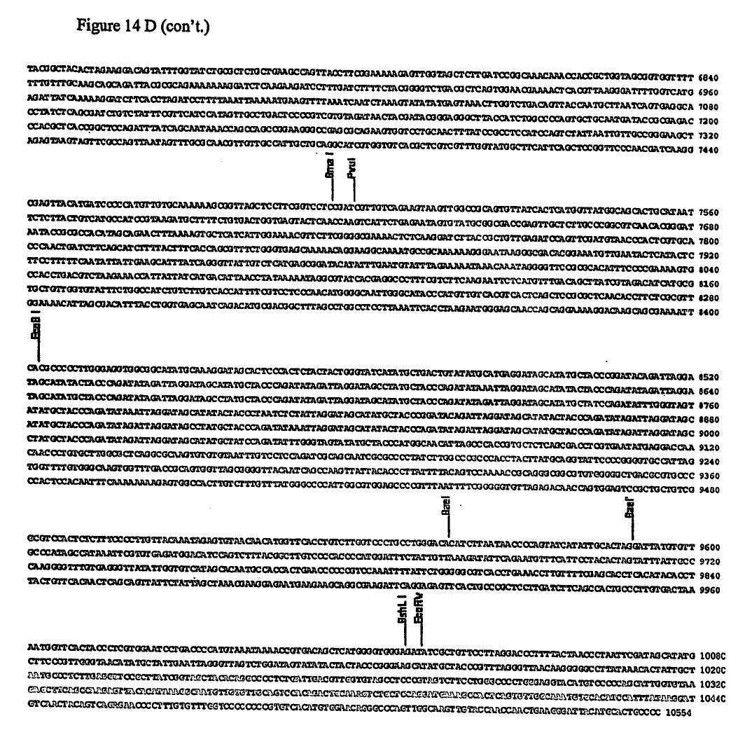

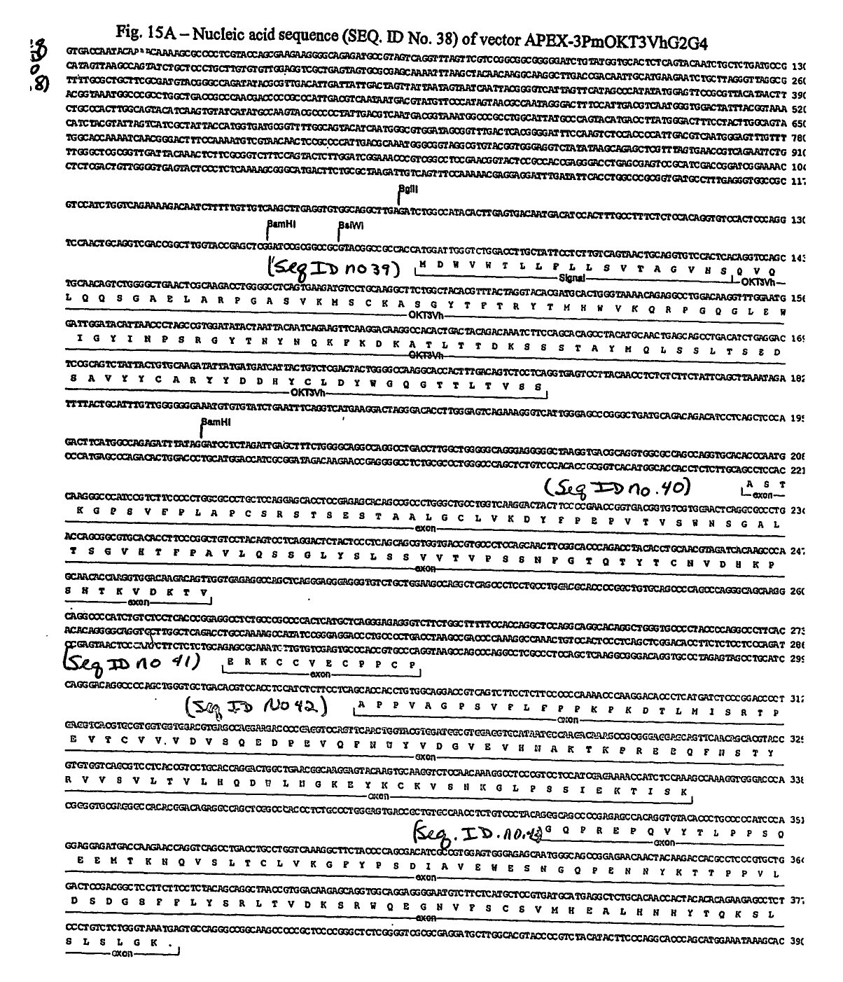

The G2/G4 constant region was excised from Puc 19 by digestion with BamHI/BglII and gel isolated. This fragment was then ligated into the expression vector APEX-3P at the BamHI site to generate APEX3PG2/G4 (see FIG. 14). The murine OKT3VH was isolated with BsiWI/BamHI digestion from the above PUC 19 vector and modified by adding a BamHI site on its 5′ end with a BamHI-BsiWI adapter. The cohesive-end adaptor duplex used to generate BamHI/BsiWI had the following sequences:

Adaptor

(Seq. ID No. 50)

5′------GATCCGCGGCCGC-----------------------3′
Adaptor

(Seq. ID NO: 51)

3′-------------------GCGCCGGCGCARTG-------5′

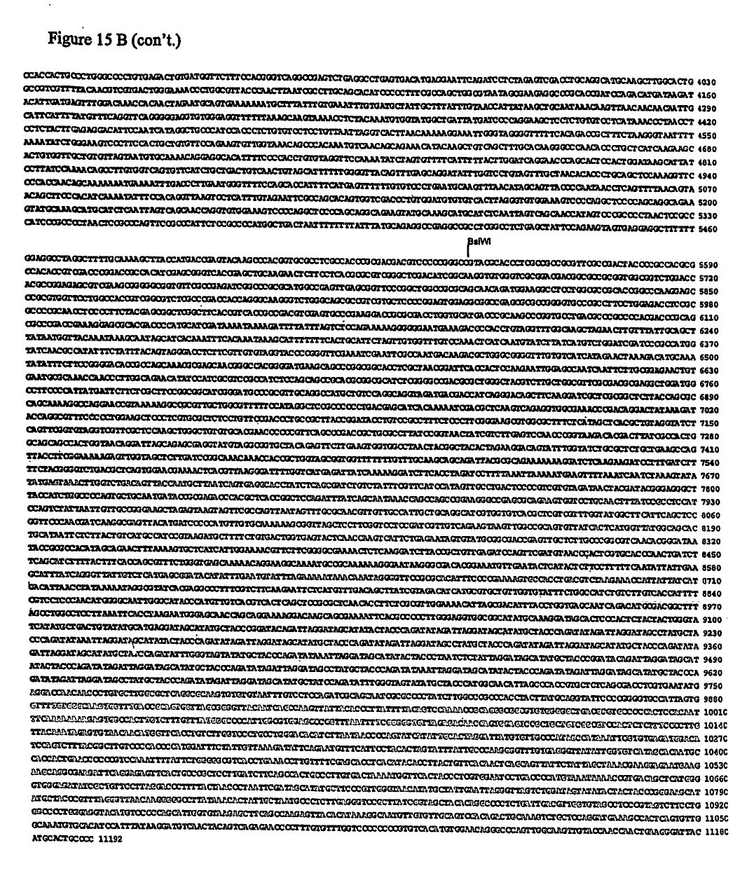

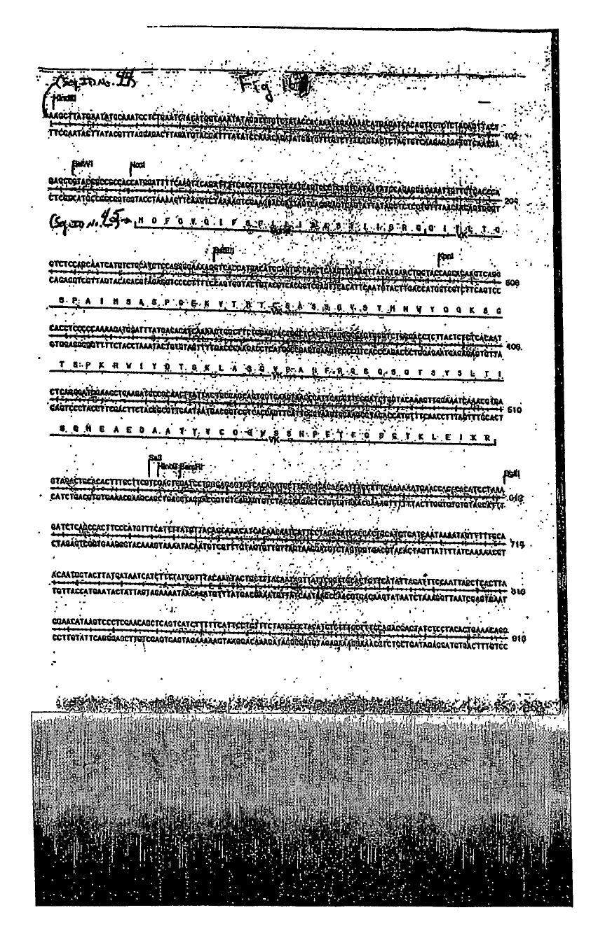

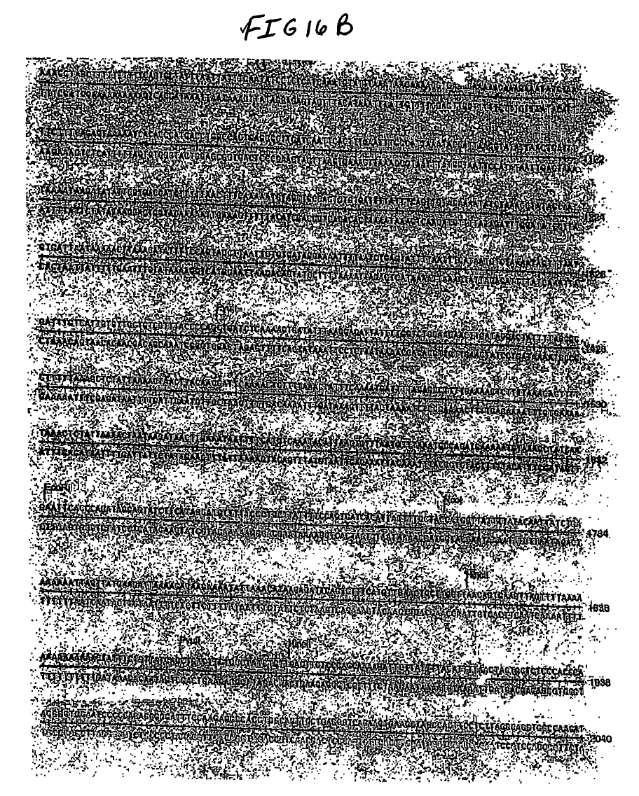

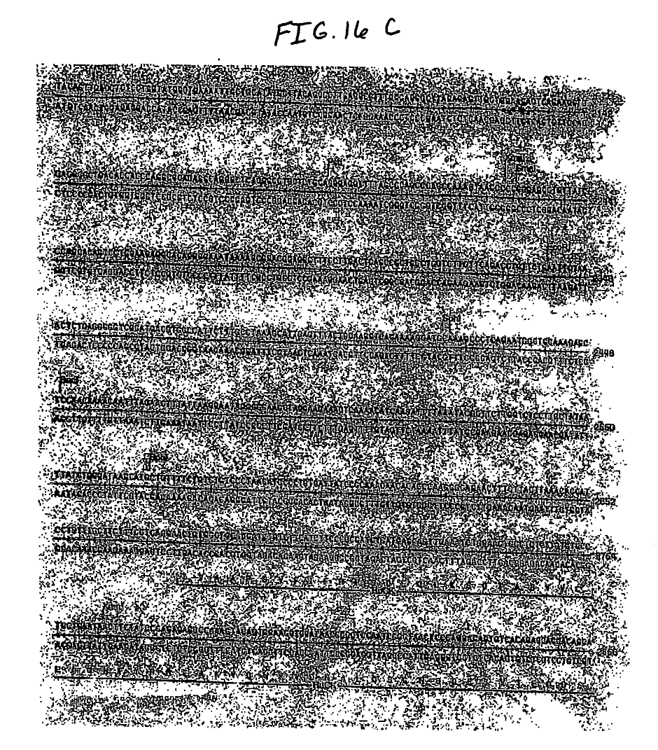

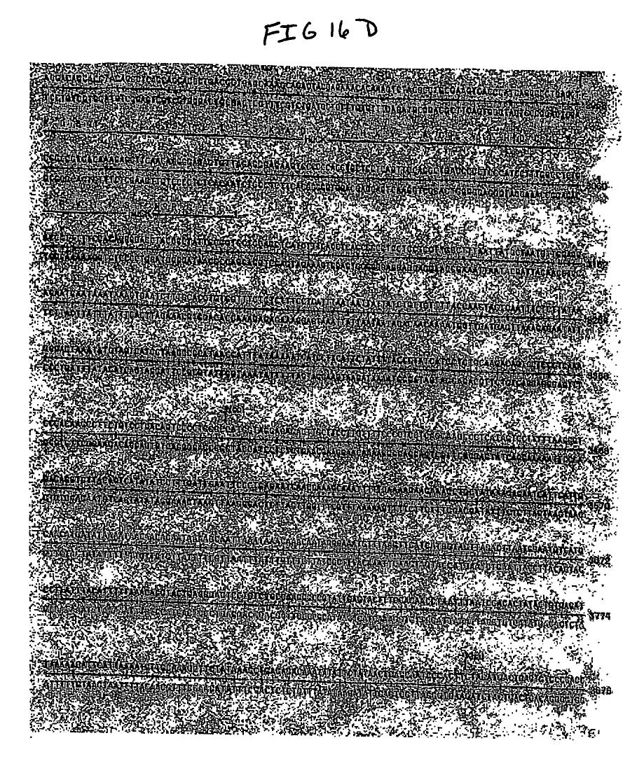

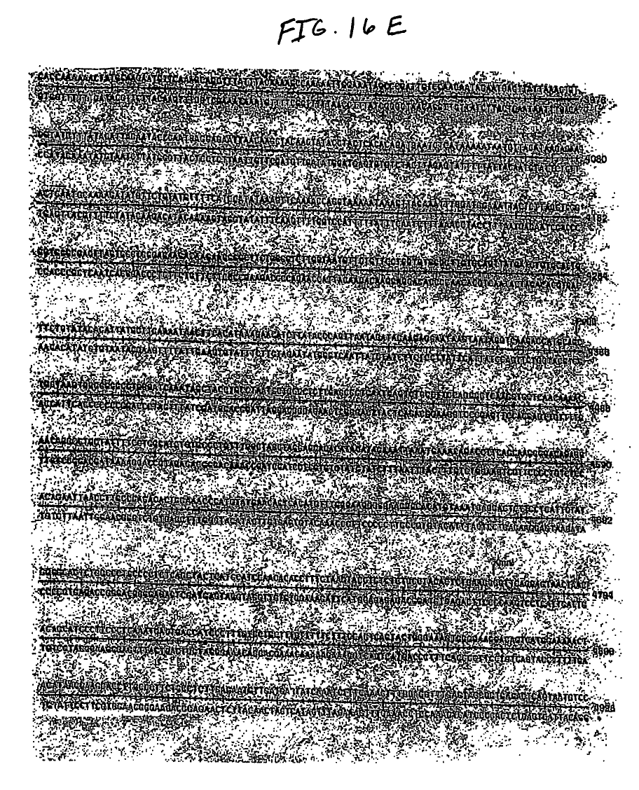

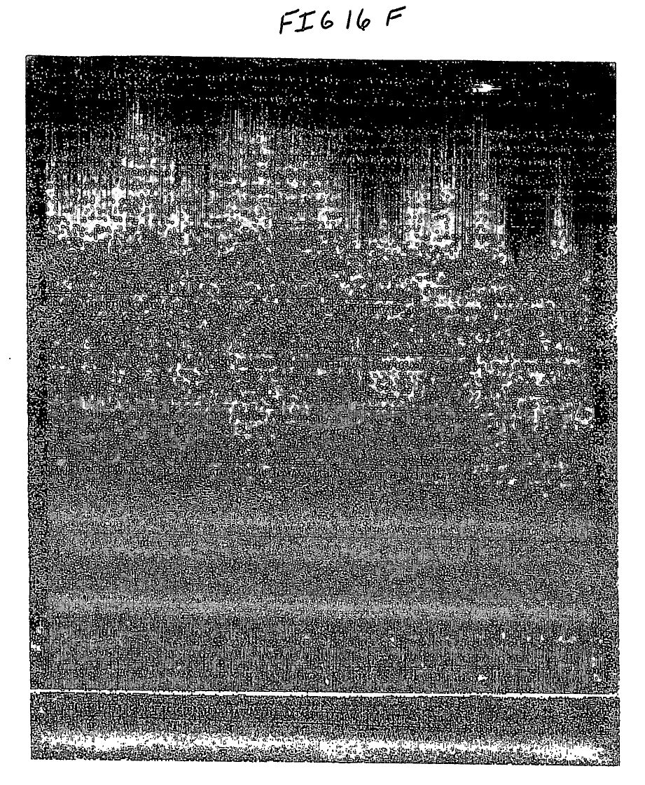

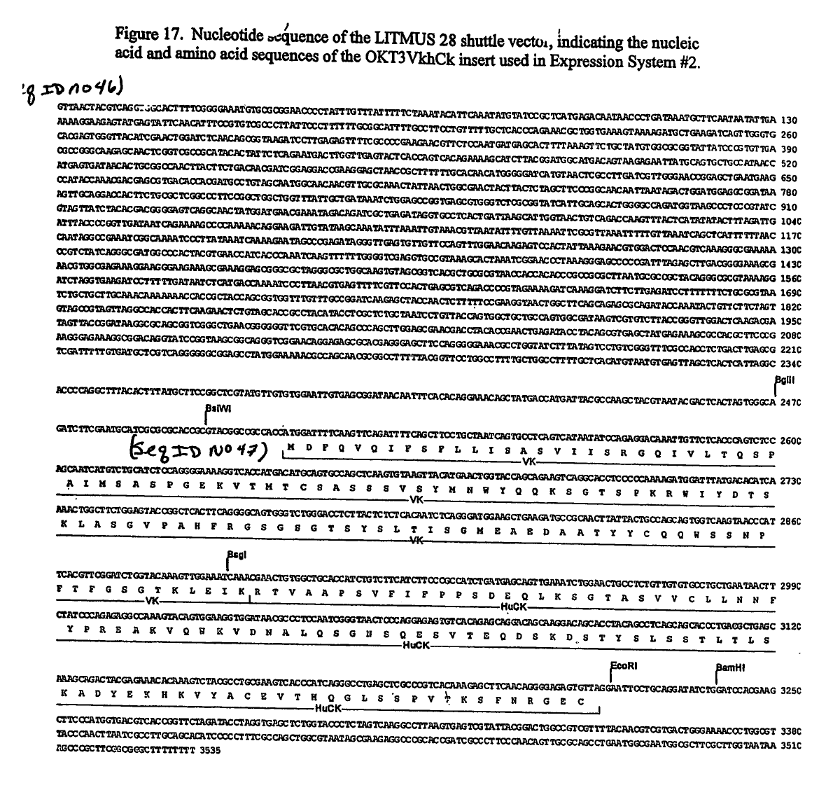

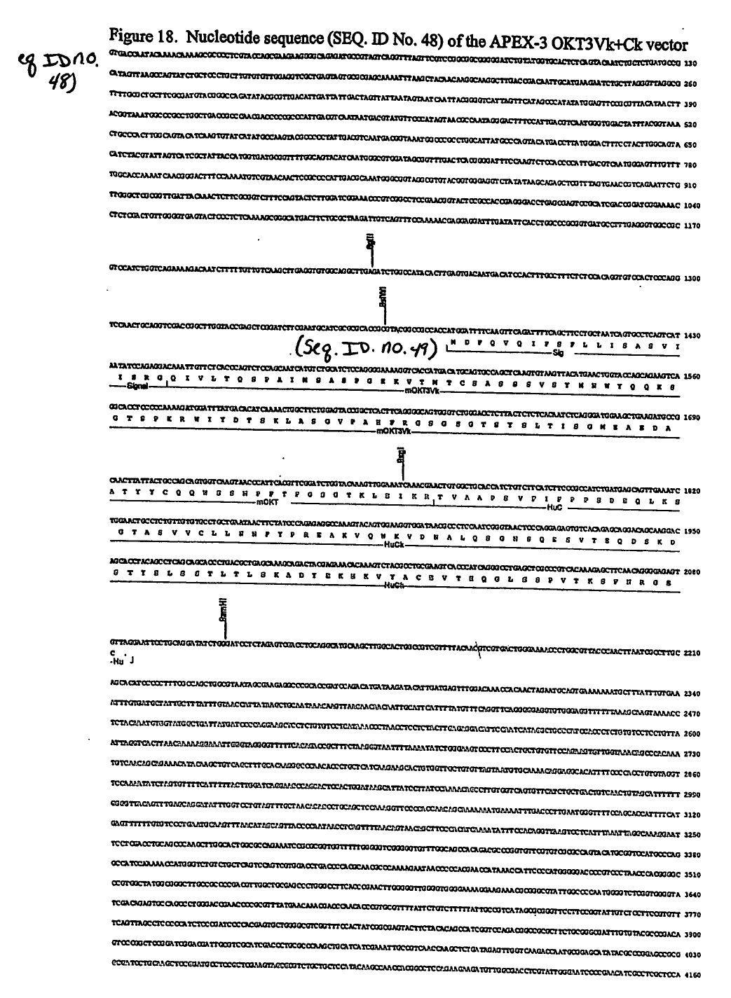

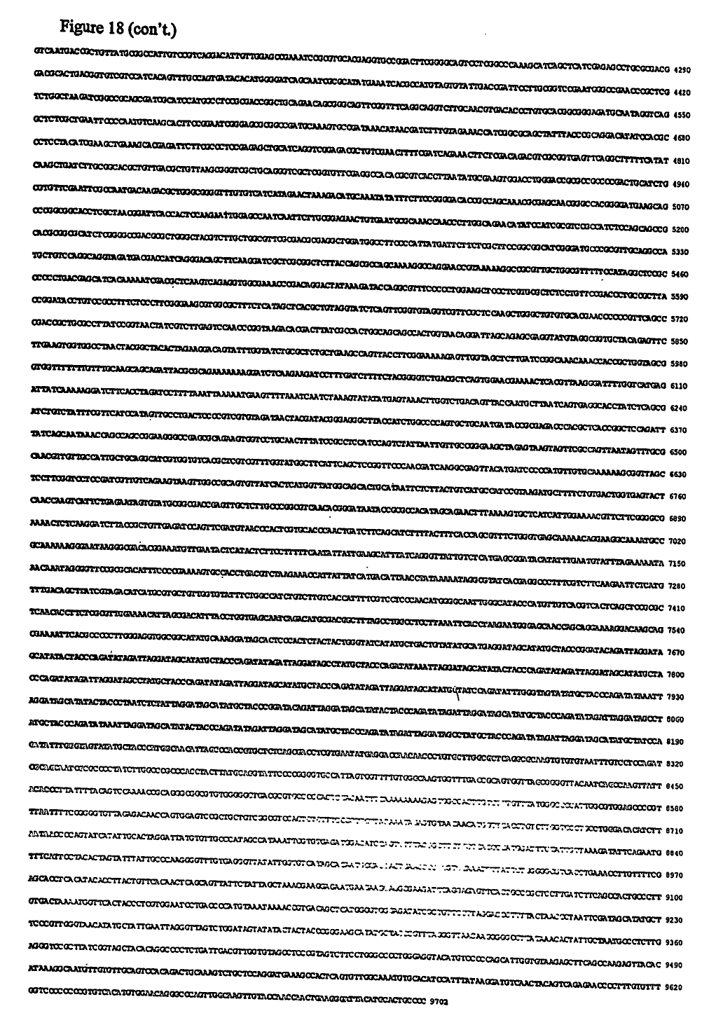

The APEX-3PG2/G4 vector was then opened with BamHI and the modified OKT3 V_{H} region inserted to generate the expression vector APEX-3PmOKT3VhG2G4 (FIG. 15). Similarly, an alternative OKT3 light chain cassette containing the original murine OKT3 VK signal sequence and variable kappa sequence (no intron) was ligated to a human kappa light chain constant region. This was accomplished by constructing OKT3 VK gene sequences by gene synthesis and ligating the sequence, as a HindIII-BamHI fragment, to human kappa constant region gene sequences previously inserted into the PUC19 cloning vector. The resulting plasmid sequence is shown in FIG. 16. Next, the OKT3VK sequence was excised from this vector with BsiWI/EcoRI, and the hCK was excised with BsgI/EcoRI from the same vector. To transfer the two fragments into the expression vector APEX-3P, a commercially available shuttle vector, LITMUS 28 was used (New England Biolabs, Inc., Beverly, Mass.). LITMUS 28 was opened with BsiWI/EcoRI digestion and ligated with the OKT3 VK fragment to generate LITMUS28mOKT3 (vector not shown), which was then opened with EcoRI/BsgI digestion and ligated to the hCK fragment above to generate LITMUS28mOKT3VKhCK (FIG. 17). The construct was then digested with BglII/BamHI to isolate the entire mOKT3VKhCK fragment. The expression vector APEX-3P was opened with BamHI and ligated with the mOKT3VKhCK fragment to generate APEX-3OKT3VK+CK (FIG. 18).
Evaluation of the Ability of the Chimeric Antibody Containing a Human G2/G4 Constant Region to Bind to the Human Receptor for IgG (FcγRI)

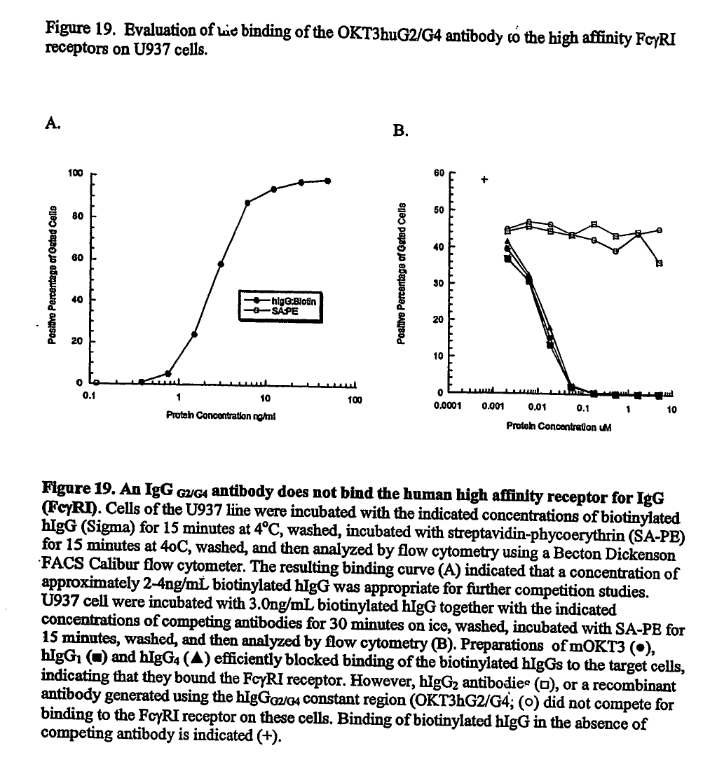

Antibodies such as OKT3, directed against the CD3 epsilon component of the T cell receptor complex, can activate human T cells by cross-linking the TCR. However, cross-linking has been shown to require accessory cells, which bind the Fc portion of the antibody through high and low affinity Fc receptors. The antibody produced in accordance with this Example was tested to evaluate binding to the human high affinity receptor for IgG (FcγRI). Cells of the U937 line were incubated with the indicated concentrations of biotinylated hIgG (Sigma) for 15 minutes at 4° C., washed, incubated with streptavidin-phycoerythrin (SA-PE) for 15 minutes at 4° C., washed, and then analyzed by flow cytometry using a Becton Dickenson FACS Calibur flow cytometer. As seen in FIGS. 19A, the resulting binding curve indicated that a concentration of approximately 2-4 ng/mL biotinylated hIgG was appropriate for further competition studies. U937 cell were incubated with 3.0 ng/mL biotinylated hIgG together with the indicated concentrations of competing antibodies for 30 minutes on ice, washed, incubated with SA-PE for 15 minutes, washed, and then analyzed by flow cytometry (FIG. 19B). Preparations of mOKT3, hIgG₁ and hIgG₄ efficiently blocked binding of the biotinylated hIgG to the target cells, indicating that they bound the FcγRI receptor. However, the recombinant chimeric antibody of this Example (containing an engineered IgG2/IgG4 human constant region) did not compete for binding to the FcγRI receptor on these cells, indicating that antibodies containing this modified constant region do not bind FcγRI.

Evaluation of Binding to the Low Affinity Human Receptor For IgG (FcγRII)

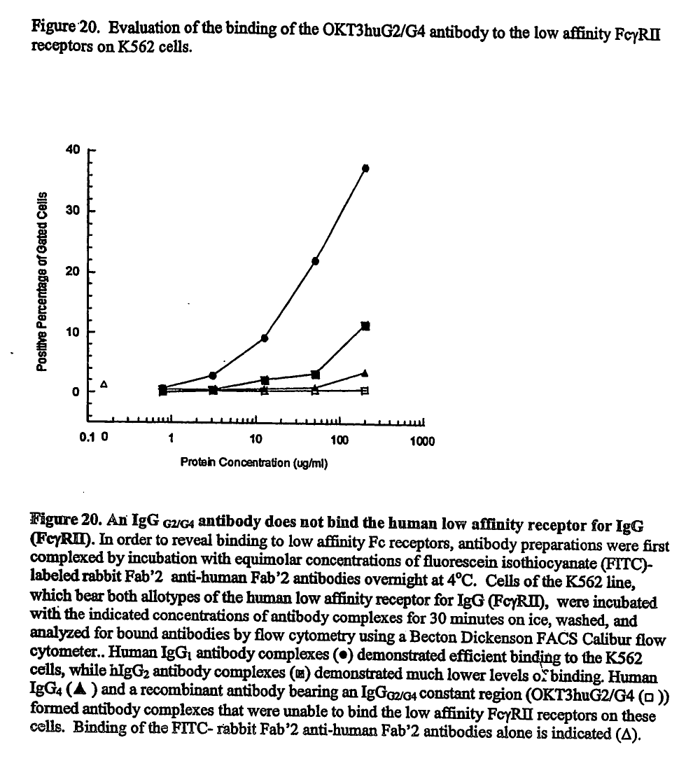

The chimeric antibody containing an engineered IgG2/IgG4 human constant region produced in accordance with this Example was tested with respect to the ability to bind the human low affinity receptor for IgG (FcγRII). In order to reveal binding to low affinity Fc receptors, antibody preparations were first complexed by incubation with equimolar concentrations of fluorescein isothiocyanate (FITC)—labeled rabbit Fab′2 anti-human Fab'2 antibodies overnight at 4° C. Cells of the K562 line, which bear both allotypes of the human low affinity receptor for IgG (FcγRII), were incubated with the indicated concentrations of antibody complexes for 30 minutes on ice, washed, and analyzed for bound antibodies by flow cytometry using a Becton Dickenson FACS Calibur flow cytometer. As seen in FIG. 20, human IgG₁ antibody complexes demonstrated efficient binding to the K562 cells, while hIgG₂ antibody complexes demonstrated much lower levels of binding. Human IgG4 and the chimeric OKT3 hG2/G4 recombinant antibody formed antibody complexes that were unable to bind the low affinity FcγRII receptors on these cells. Binding of the FITC—rabbit Fab′2 anti-human Fab′2 antibodies alone is also indicated.

Evaluation of Ability to Induce Cytokine Production in PBL

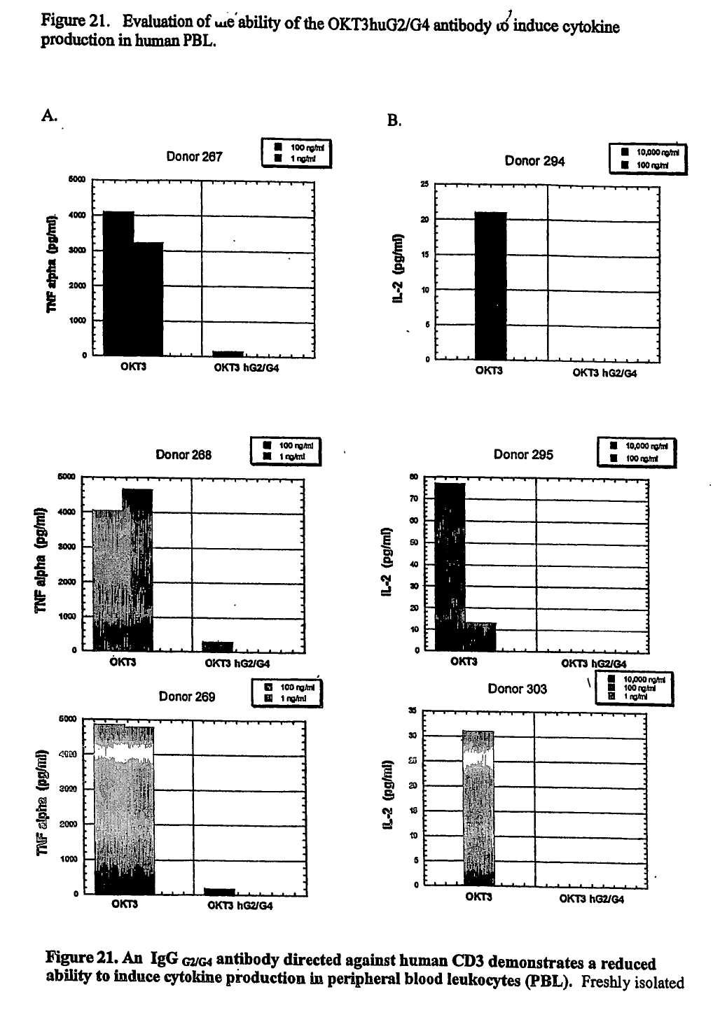

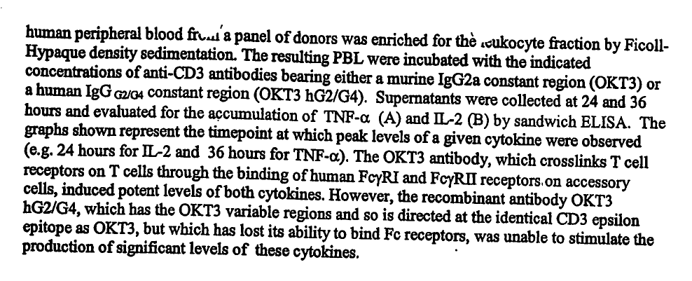

A chimeric antibody of Example 1 directed against human CD3 and containing an engineered IgG2/IgG4 human constant region was evaluated for its ability to induce cytokine production in peripheral blood leukocytes (PBL). Freshly isolated human peripheral blood from a panel of donors was enriched for the leukocyte fraction by Ficoll-Hypaque density sedimentation. The resulting PBL were incubated with the indicated concentrations of anti-CD3 antibodies bearing either a murine IgG2a constant region (OKT3) or a human IgG_{G2/G4} constant region (OKT3 hG2/G4) (see, FIG. 21). Supernatants were collected at 24 and 36 hours and evaluated for the accumulation of TNF-α (A), and IL-2 (B) by sandwich ELISA. The graphs shown in FIG. 21 represent the time point at which peak levels of a given cytokine were observed (e.g. 24 hours for IL-2 and 36 hours for TNF-α). The OKT3 antibody, which binds both human FcγRI and FcγRII receptors, induced potent levels of both cytokines. However, the chimeric recombinant antibody OKT3 hG2/G4, which binds to the same CD3 epsilon epitope as OKT3 but which has lost its ability to bind Fc receptors, were unable to stimulate the production of significant levels of any of the cytokines examined.

Evaluation of Ability to Activate Target T Cells from Human PBL

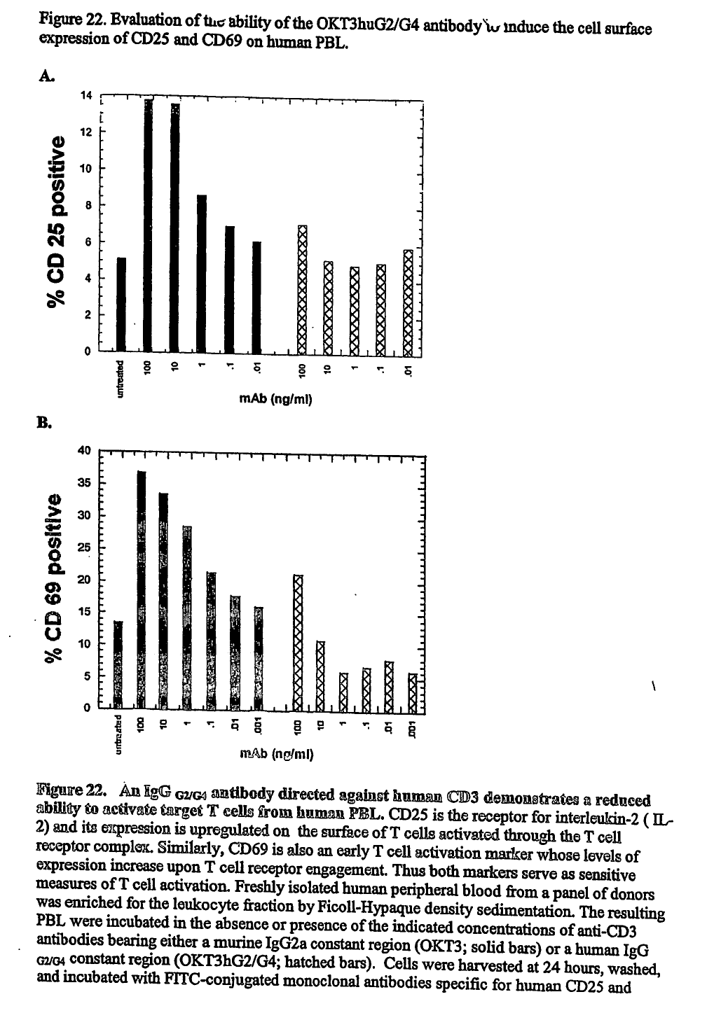

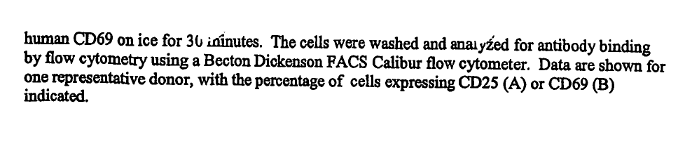

A chimeric antibody of Example 1 directed against human CD3 and containing an engineered IgG2/IgG4 human constant region was evaluated for the ability to activate target T cells from human PBL. CD25 is the receptor for interleukin-2 (IL-2) and its expression is upregulated on the surface of T cells activated through the T cell receptor complex. Similarly, CD69 is also an early T cell activation marker whose levels of expression increase upon T cell receptor engagement. Thus both markers serve as sensitive measures of T cell activation. Freshly isolated human peripheral blood from a panel of donors was enriched for the leukocyte fraction by Ficoll-Hypaque density sedimentation. The resulting PBL were incubated in the absence or presence of the indicated concentrations of anti-CD3 antibodies bearing either a murine IgG2a constant region (OKT3) or a human IgG_{G2/G4} constant region (OKT3 hG2/G4); see FIG. 22). Cells were harvested at 24 hours, washed, and incubated with FITC-conjugated monoclonal antibodies specific for human CD25 and human CD69 on ice for 30 minutes. The cells were washed and analyzed for antibody binding by flow cytometry using a Becton Dickenson FACS Calibur flow cytometer. Data are shown in FIGS. 22A and 22B for one representative donor, with the percentage of cells expressing CD25 (A) or CD69 (B) indicated.

### Generation and Expression of a Human L-SIGN-Fc Fusion Protein with Human G2G4 Fc Portion

A human L-SIGN-human G2G4 fusion protein is generated by fusing two PCR fragments derived from cDNA coding for the human L-SIGN (as the non Fc component) and the Fc portion of human immunoglobulin HuG2G4. Oligonucleotides P1, cagatgtgatatcTCCAAGGTCCCCAGCTCCCTAAG (SEQ ID NO: 52), and P2, tgggctcgagTTCGTCTCTGMGCAGGCTGCG (SEQ ID NO: 53), are used to amplify the extracellular portion of hL-SIGN from human spleen cDNA library (The regions of the primer that are complementary to the human L-SIGN are indicated with capital letters). P1 contains an upstream EcoRV restriction endonuclease site to fuse with a leader sequence (KLV56). In P2, a downstream XhoI restriction endonuclease site is used to fuse with the hG2G4 Fc region. The primers P3, agacgaactcGAGCGC AAATGTTGTGTCGAGT (SEQ ID NO: 54), and P4, cggccctggcactcaTTTACCCAGA GACAGGGAGAGGCT (SEQ ID NO: 55), are used to amplify the human G2G4 hybrid Fc region from Glu⁹⁹ of the hinge domain to the carboxyl terminus by using a plasmid containing the human G2G4 constant region. Capital letters indicates complementary regions to the human G2G4 sequence. In P3, an upstream XhoI restriction endonuclease site is designed to ligate with hL-SIGN. P4 contains downstream sequences for one stop codon and NgoMIV restriction endonuclease site. The PCR amplified human L-SIGN and human G2G4 Fc region fragments are TA cloned into pCR2.1 vector and accurate sequence confirmed. The resulting plasmid pCR2.1 hL-SIGN is digested with EcRV/XhoI, and the plasmid pCR2.1hG2G4 is digested with XhoINgoMIV. The resulting L-SIGN and hG2G4 fragments are ligated into a modified Apex3P plasmid (Alexion Pharmaceuticals, Inc.). EcoRV/NgoMIV contains a KLV56 leader with a Kozak sequence and ATG corresponding to the codon for the initiating methionine (5′-CGCCCTTCCACC ATGGACATGAGGGTCCCCGCTCAGCTCCTGGGGCTC CTGCTACTCTGGCTCCGAGGTGCCAGATGT-3′ (SEQ ID NO: 56)).

Cell Culture and Protein Purification

293 EBNA human embryonic kidney cells transfected with Apex3P-hL-SIGNhG2G4 are grown in DMEM (Cellgro #10-013-CV) with 10% heat inactivated FBS, 100 lU/ml penicillin, 100 □g/ml streptomycin, 2 mM glutamine with 250 ug/ml G418 Sulfate and 1 ug/ml puromycin. Cells are grown and selected at 37° C. and 5% CO₂. Confluent T-175 flasks of selected cells are washed with 15 ml HBSS in order to remove serum proteins before the addition of 30 ml IS Pro serum free medium (Irvine Scientific, Santa Ana, Calif., Catalog # 91103) supplemented with L-Glutamine (amount noted on bottle) and penicillin/streptomycin to each flask. Two to three day supernatants are concentrated and purified by Protein A chromatography.

Throughout this specification, various publications and patent disclosures are referred to. The teachings and disclosures thereof, in their entireties, are hereby incorporated by reference into this application to more fully describe the state of the art to which the present invention pertains.

Although preferred and other embodiments of the invention have been described herein, further embodiments may be perceived by those skilled in the art without departing from the scope of the invention as defined by the following claims.

We claim:

1. A method for reducing antibody-mediated cell activation or inflammation events comprising administering an antibody which binds to either a cell surface molecule or a soluble molecule that binds to a cell surface molecule, the antibody including an engineered heavy chain constant region having a first portion derived from one or more human IgG2 antibodies and a second portion derived from one or more human IgG4 antibodies.

2. A method as in claim 1 wherein at least the CH1 and hinge regions are derived from one or more human IgG2 antibodies and at least a portion of the CH2 and CH3 regions are derived from one or more human IgG4 antibodies.

3. A method as in claim 1 wherein the antibody binds to a human complement component.

4. A method as in claim 1 wherein the antibody binds to a cytokine receptor.

5. A method as in claim 1 wherein the antibody binds to an adhesion molecule.

6. A method as in claim 1 wherein the antibody binds to a cell differentiation antigen.

7. A method as in claim 1 wherein the antibody binds to a cell activation antigen.

8. A method as in claim 1 wherein the antibody binds to a soluble molecule that binds to cell surface molecules.

9. A method as in claim 1 wherein the antibody binds to a cytokine.

10. A method as in claim 1 wherein the antibody binds to a chemokine.

11. A method as in claim 1 wherein the antibody binds to a growth factor.

12. A method as in claim 1 wherein the antibody binds to a molecule that induces cell differentiation.

13. A method as in claim 1 wherein the antibody binds to a molecule that induces cell activation.

14. A method as in claim 1 wherein the antibody binds to a cell surface molecule.

15. A method for preventing or reducing cytokine release comprising administering an antibody which binds to either a cell surface molecule or a soluble molecule that binds to a cell surface molecule, the antibody including an engineered heavy chain constant region having a first portion derived from one or more human IgG2 antibodies and a second portion derived from one or more human IgG4 antibodies.

16. A method for preventing or reducing the severity of cytokine release syndrome comprising administering an antibody which binds to either a cell surface molecule or a soluble molecule that binds to a cell surface molecule, the antibody including an engineered heavy chain constant region having a first portion derived from one or more human IgG2 antibodies and a second portion derived from one or more human IgG4 antibodies.

17. A fusion protein comprising a non-Fc component and an Fc region having a first portion derived from one or more human IgG2 antibodies and a second portion derived from one or more human IgG4 antibodies.

18. The fusion protein of claim 17 wherein the Fc region includes at least a portion of a CH₁ region.

19. The fusion protein of claim 17 wherein the Fc region does not include any portion of a CH₁ region.

20. The fusion protein of claim 17 wherein the Fc region includes at least a part of a hinge region.

21. The fusion protein of claim 17 wherein the non-Fc component comprises an antibody fragment.

22. The fusion protein of claim 17 wherein the non-Fc component is a member selected from the group consisting of single chain, scFv, f(ab) and F(ab)′₂.

23. The fusion protein of claim 17 wherein the non-Fc component comprises a variable region of an antibody having a mimetic peptide inserted within or in place of at least a portion of at least one CDR.

24. The fusion protein of claim 17 wherein the non-Fc component comprises a peptide.

25. The fusion protein of claim 17 wherein the non-Fc component comprises a protein or fragment thereof.

26. The fusion protein of claim 17 wherein the non-Fc component comprises a member selected from the group consisting of cytokines, hormones, enzymes, ligands, growth factors, receptors and antibody fragments.

27. The fusion protein of claim 17 wherein the non-Fc component comprises a member selected from the group consisting of peptides displaying G-CSF activity, peptides displaying GHR activity, peptides displaying prolactin activity, nerve growth factor mimetics, IL-2 mimetics, glucogon-like peptide-1, tetrapeptide I (D-lysine-L-asparaginyl-L-prolyl-L-tyrosine), N-terminal peptide of vMIP-II, antagonist peptide ligand (AFLARM) of the thrombin receptor, peptide CGRP, receptor antagonist CGRP, parathyroid hormone (PTH)-1 receptor antagonist, acid fibroblast growth factor binding peptide, human brain natriuretic peptide (hBNP), exendin-4, GLP-1 (7-36), GPL-2 (1-34), glucagons, PACAP-38, platelet-derived growth factor (PDGF), keratinocyte growth factor (KGF), epidermal growth factor (EGF), vascular endothelial growth factor (VEGF), insulin, nerve growth factor (NGF), insulin-like growth factor (IGF), transforming growth factor (TGF), hepatic growth factor (HGF), fibroblast growth factor (FGF), the product of the Wnt-2 proto-oncogne (wnt-2) and the binding domain of human cytotoxic T-lymphocyte-associated antigen 4.

28. A method of increasing the half life of a non-Fc component by fusing the non-Fc component to an Fc region having a first portion derived from one or more human IgG2 antibodies and a second portion derived from one or more human IgG4 antibodies.

29. A method of increasing the avidity of a non-Fc component for a molecule to which the non-Fc component binds by fusing the non-Fc component to an Fc region having a first portion derived from one or more human IgG2 antibodies and a second portion derived from one or more human IgG4 antibodies.

30. A method of forming a dimer of a non-Fc component by fusing the non-Fc component to an Fc region having a first portion derived from one or more human IgG2 antibodies and a second portion derived from one or more human IgG4 antibodies.

31. A method of facilitating purification of a non-Fc component by fusing the non-Fc component to an Fc region having a first portion derived from one or more human IgG2 antibodies and a second portion derived from one or more human IgG4 antibodies.

32. A method of reducing or eliminating Fc receptor binding and complement activation associated with a Fc fusion protein comprising fusing a non-Fc component to an Fc region having a first portion derived from one or more human IgG2 antibodies and a second portion derived from one or more human IgG4 antibodies.

33. A method as in claim 32 wherein the Fc receptor binding and complement activation is reduced or eliminated in vitro.

34. A method of improving expression of a non-Fc component in mammalian cells by creating a Fc fusion protein having the non-Fc component fused to an Fc region having a first portion derived from one or more human IgG2 antibodies and a second portion derived from one or more human IgG4 antibodies.

35. The fusion protein of claim 17 wherein the Fc region is attached to an amino terminus of the non-Fc component.

36. The fusion protein of claim 17 wherein the Fc region is attached to the carboxy terminus of the non-Fc component.

37. Nucleic acid encoding a fusion protein in accordance with claim 17.

38. Nucleic acid as in claim 37 which includes introns.

39. Nucleic acid as in claim 37 which does not include introns.

40. An expression vector containing nucleic acid in accordance with claim 37.

41. A host cell transfected with an expression vector in accordance with claim 39.

42. A composition comprising a fusion protein in accordance with claim 17 and a pharmaceutically acceptable carrier.
